(19) United States
(12) Patent Application Publication
(10) Pub. No.: US 2023/0092648 A1
(43) Pub. Date: Mar. 23, 2023

(54) COMBINATION CONTAINING A DUOCARMYCIN DERIVATIVE-COMPRISING ANTIBODY-DRUG CONJUGATE AND THIOSULFATE

(71) Applicant: BYONDIS B.V., Nijmegen (NL)

(72) Inventors: Johannes Henricus Matthias SCHELLENS, Nijmegen (NL); Rudy Gerardus Elisabeth COUMANS, Nijmegen (NL)

(21) Appl. No.: 17/797,812

(22) PCT Filed: Feb. 3, 2021
(86) PCT No.: PCT/EP2021/052509

§ 371 Date: Aug. 5, 2022

(30) Foreign Application Priority Data
Feb. 6, 2020 (EP) 20155842.6

(51) Int. Cl. A61K 47/68; A61P 35/00; A61K 33/04; A61K 9/00

(52) U.S. Cl. CPC A61K 47/6803; A61K 47/6851; A61K 47/6889; A61P 35/00; A61K 33/04; A61K 9/0048; A61K 9/0019

(57) ABSTRACT

The present invention relates to the combined use of a duocarmycin derivative-comprising antibody-drug conjugate and thiosulfate in the treatment of a tumor in a human, whereby the thiosulfate prevents or reduces unwanted non-target tissue toxicity of the antibody-drug conjugate.

### FIELD OF THE INVENTION

The present invention relates to the combined use of a duocarmycin derivative-comprising antibody-drug conjugate and thiosulfate in the treatment of a tumor in a human, whereby the thiosulfate prevents or reduces unwanted non-target tissue toxicity of the antibody-drug conjugate.

### BACKGROUND OF THE PRESENT INVENTION

Duocarmycins are members of a family of antitumor antibiotics that include duocarmycin A, duocarmycin SA, and CC-1065. They are known for their potent antitumor properties, but are normally not used on their own because of their extremely high toxicity. Currently, duocarmycins are being explored as cytotoxic drugs in antibody-drug conjugates (ADCs).

ADCs have the potential to address the great unmet need for effective new treatments in cancer by directing the highly potent cytotoxic drug specifically to cancer cells, thereby enhancing efficacy while reducing the potential systemic toxic side effects of the small molecule drug.

Linker-drug vc-seco-DUBA

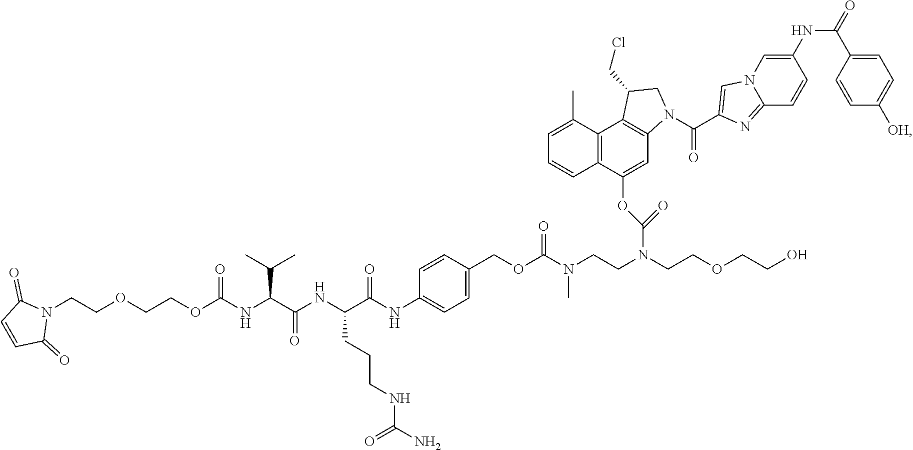 [SMILES: Cc1cccc2c(OC(=O)N(CCOCCO)CCN(C)C(=O)OCc3ccc(NC(=O)[C@H](CCCNC(N)=O)NC(=O)[C@@H](NC(=O)OCCOCCN4C(=O)C=CC4=O)C(C)C)cc3)cc3c(c12)[C@H](CCl)CN3C(=O)c1cn2cc(NC(=O)c3ccc(O)cc3)ccc2n1]

first disclosed in WO2011/133039 as compound 18b on p. 210, 11. 21-27, is an example of a highly potent CC-1065 analogue. The ADC of vc-seco-DUBA with the anti-HER2 antibody trastuzumab, i.e., SYD985 or (vic-)trastuzumab duocarmazine, was used successfully in several preclinical studies (van der Lee et al., Molecular Cancer Therapeutics, 2015, 14(3), 692-703; Black et al., Molecular Cancer Therapeutics, 2016, 15(8), 1900-1909) and Phase I clinical trials (ClinicalTrials.gov NCT02277717). Trastuzumab duocarmazine is currently being tested in the TULIP Phase III clinical trial in patients with HER2-positive locally advanced or metastatic breast cancer (ClinicalTrials.gov NCT03262935).

As with other drugs, the use of trastuzumab duocarmazine is related with unwanted non-target tissue toxicity. For example, ocular toxicity and local toxicity caused by extravasation of the ADC-containing solution during intravenous infusion were observed during the Phase I clinical trial (Banerji et al., Lancet Oncology, 2019, 20, 1124-1135). This unwanted non-target tissue toxicity may be aspecific, i.e., caused by premature release of the toxic drug before binding to the target, through diffusion of released toxic drug from a tumor cell into surrounding tissue, the so-called bystander effect, or through aspecific uptake of the ADC into a cell, e.g. macropinocytosis. Alternatively, non-target tissue toxicity may be antigen-mediated by binding of the antibody (i.e., trastuzumab) to the antigen-target (i.e., HER2) expressed on cells of non-tumor tissue and subsequent internalization of the ADC and intracellular release of the cytotoxic drug.

Recently, a first-in-human trial of another duocarmycin derivative-comprising anti-5T4 ADC (i.e., SYD1875) was started (ClinicalTrials.gov NCT04202705).

Hence, there is a need for preventing and/or reducing unwanted non-target tissue toxicity of duocarmycin derivative-comprising ADCs in general and of vc-seco-DUBA-comprising ADCs in particular.

### BRIEF DESCRIPTION OF THE PRESENT INVENTION

The present invention relates to the combined use of a duocarmycin derivative-comprising ADC and thiosulfate in the treatment of a tumor in a human, whereby the thiosulfate prevents or reduces unwanted non-target tissue toxicity of the ADC.

In a first aspect, the present invention relates to an ADC for use in the treatment of a tumor in a human, wherein the ADC is administered in combination with thiosulfate and wherein the ADC is a compound of formula (I)

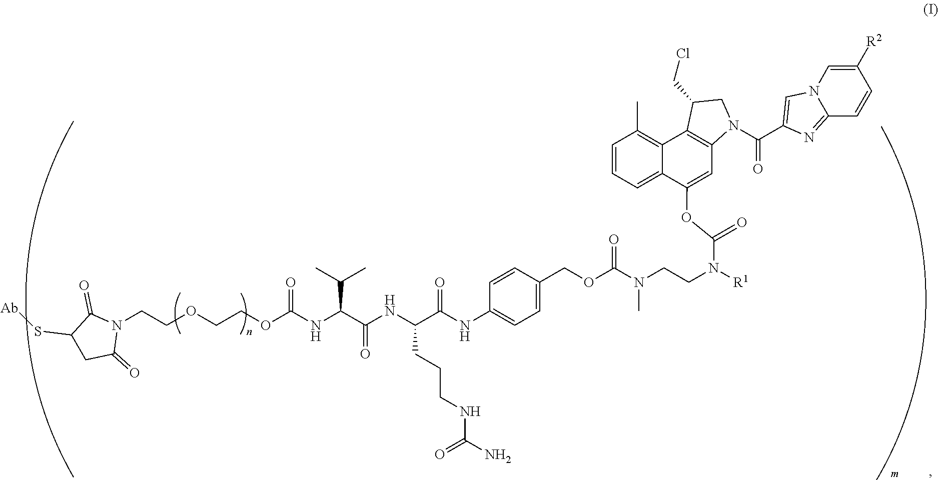

wherein
Ab is an antibody or an antigen-binding fragment of an antibody;
n is0, 1,2or3;
m represents an average drug-to-antibody ratio (DAR) of from 1 to 6;
R¹ is selected from the group consisting of

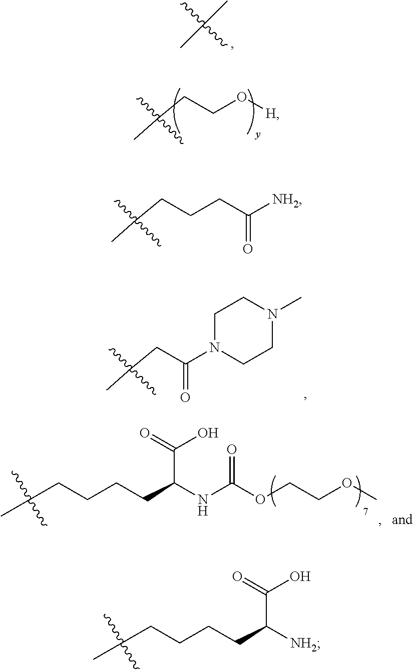

y is an integer of from 1-16; and
R² is selected from the group consisting of

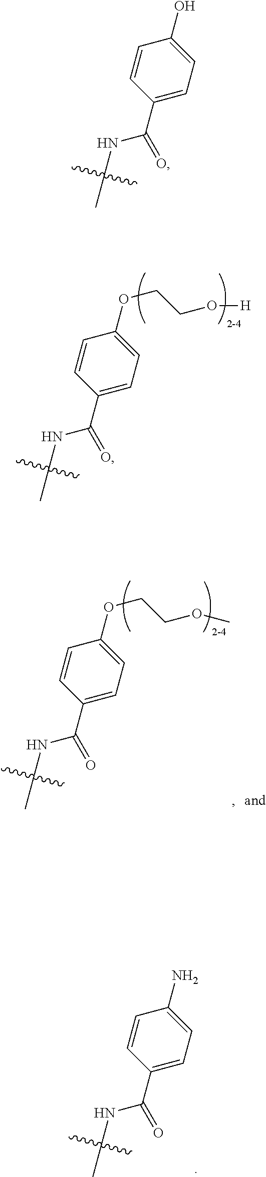

In a specific embodiment, the ADC is a compound of formula (II)

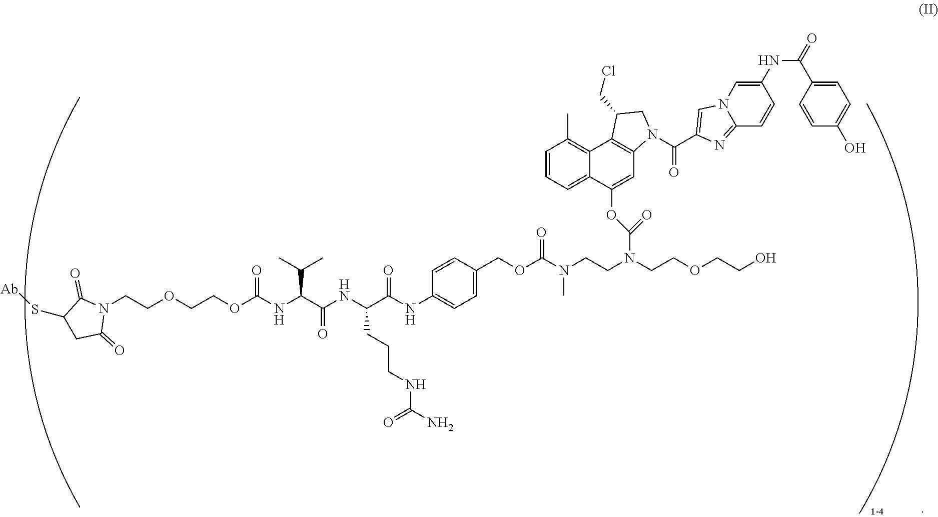

In a second aspect, the present invention relates to a composition comprising thiosulfate for use in a human in the prevention or reduction of toxicity associated with the administration to the human of an ADC of formula (I) or (II).

In a third aspect, the present invention relates to the use of an ADC of formula (I) or (II) in the manufacture of a medicament for use in combination therapy for treatment of a tumor in a human, wherein the medicament is administered in combination with thiosulfate.

In a fourth aspect, the present invention relates to a product containing an ADC of formula (I) or (II) and thiosulfate as a combined preparation for the simultaneous, separate or sequential use in the treatment of a tumor in a human.

In a fifth aspect, the present invention relates to a method for preventing or reducing toxicity associated with the administration of an ADC of formula (I) or (II) comprising administering an effective amount of the ADC in combination with an effective amount of thiosulfate, wherein the thiosulfate is administered from about three weeks before to about 1 hour after the first administration of the ADC and the administration of thiosulfate is repeated at regular intervals until up to three months after the last administration of the ADC.

### BRIEF DESCRIPTION OF THE DRAWINGS

FIG. 1. Rearrangement of a seco compound to a cyclopropyl-containing compound.

FIG. 2. Detoxification of a cyclopropyl-containing compound by thiosulfate.

FIG. 3A. UV chromatograms of SYD986 (1 pM) blank solution and SYD986 (1 pM) +STS (10 mM) dissolved in acetonitrile/water (1:1).

FIG. 3B. MS analysis results of the SYD986 blank (upper panel) and the reaction product of SYD986 and STS (lower panel).

FIG. 4A. Viability of SK-BR-3 cells after exposure to various concentrations of SYD986 or various concentrations of SYD986+1 mM STS.

FIG. 4B. Viability of SK-BR-3 cells after exposure to various concentrations of SYD1875 or various concentrations of SYD1875+1 mM STS.

### DETAILED DESCRIPTION OF THE PRESENT INVENTION

Duocarmycins are a class of structurally related toxins first isolated from a culture broth of Streptomyces species. They are members of a family of antitumor antibiotics that include duocarmycin A, duocarmycin SA, and CC-1065. Duocarmycins bind to the minor groove of DNA and subsequently cause irreversible alkylation of DNA. This disrupts the nucleic acid architecture, which eventually leads to tumor cell death.

Because of their extremely high toxicity, duocarmycins, as well as their synthetic derivatives, are normally not used on their own, but used e.g., as cytotoxic drugs in ADCs.

Trastuzumab duocarmazine, an ADC consisting of the antibody trastuzumab and duocarmycin derivative linker-drug vc-seco-DUBA. of formula (III)

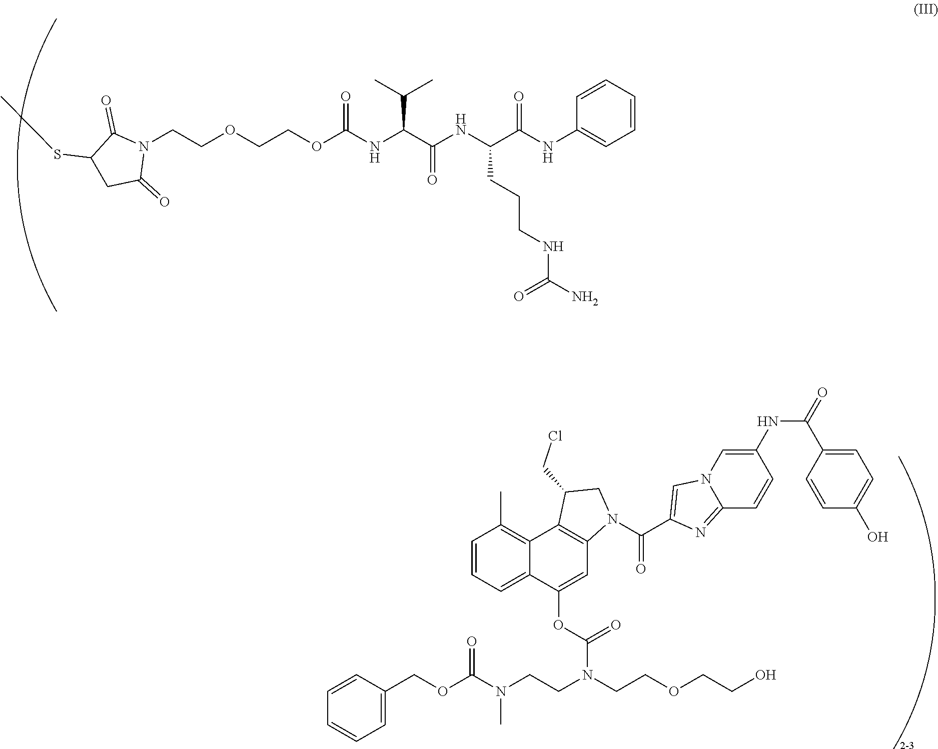 [SMILES: CSC1CC(=O)N(CCOCCOC(=O)N[C@H](C(=O)N[C@@H](CCCNC(N)=O)C(=O)Nc2ccccc2)C(C)C)C1=O ; Cc1cccc2c(OC(=O)N(CCOCCO)CCN(C)C(=O)OCc3ccccc3)cc3c(c12)[C@H](CCl)CN3C(=O)c1cn2cc(NC(=O)c3ccc(O)cc3)ccc2n1]

is currently being tested in the TULIP Phase III clinical trial (ClinicalTrials.gov NCT03262935). A concern regarding the development of trastuzumab duocarmazine and PGP-²⁶,C1 other duocarmycin derivative-comprising ADCs, including SYD1875, is that the cytotoxic activity and subsequent effectiveness may be associated with substantial toxicity for some patients. In Phase I clinical studies, e.g., ocular toxicity and local toxicity caused by extravasation of the ADC during intravenous infusion were observed. Both trastuzumab duocarmazine (SYD985) and SYD1875 are ADCs according to formula (I) and (II).

The term “unwanted non-target tissue toxicity” as used herein means the toxicity towards any tissue other than the target tissue, i.e., toxicity towards non-tumor tissue, preferably the toxicity towards healthy cells. Unwanted non-target tissue toxicity may be aspecific or antigen-mediated.

Aspecific unwanted non-target tissue toxicity may occur for example by premature release of the toxic drug from the ADC, through bystander effect, or through aspecific uptake of the ADC into a cell.

An example of premature release of the toxic drug from the ADC is cleavage of the linker-drug from the ADC while in circulation or after extravasation, i.e., leakage of intravenously infused ADC into the extravascular tissue around the site of infusion. Cleavage of the linker-drug from the ADC results in formation of the active toxin which may cause adverse events such as for example tissue necrosis. Bystander effect is the event wherein cells that have not bound and/or processed an ADC in the proximity of a cell which has bound and processed an ADC, are killed. Without wishing to be bound by any theory, it is believed that the drug is released by a dying cell, thereby killing other cells in its proximity. The bystander effect may occur in the tumor affecting other tumor cells in the proximity of the ADC-affected tumor cell, where it is a desired effect. However, it may also occur inside or outside the tumor, whereby non-tumor cells located in the proximity of an ADC-affected (non-)tumor cell are killed. In the present invention, the term bystander effect is limited to the situation where it occurs outside the tumor and affects non-tumor cells and not in the proximity of the tumor. An example of aspecific uptake is macropinocytosis. Macropinocytosis is a means by which eukaryotic cells ingest extracellular liquid and dissolved molecules. After uptake of the ADC into the cell, the cytotoxic drug is released intracellulary resulting in cell death.

Unwanted non-target tissue toxicity may also be antigen-mediated, i.e., in cases where the antigen is also present on other tissues than the target tissue. Without wishing to be bound by any theory, it is thought that the antibody in the ADC binds to the antigen-target expressed on cells of non-tumor tissue, the ADC is subsequently internalized, followed by intracellular release of the cytotoxic drug and cell death.

Surprisingly and unexpectedly, the present inventors found that thiosulfate can detoxify the active cyclopropyl-containing duocarmycin drug as released from the ADC (or prematurely released linker-drug) and can as such prevent and/or reduce unwanted non-target tissue toxicity. The present invention thus relates to the combined use of a duocarmycin derivative-comprising ADC and thiosulfate.

Therefore, in one aspect, the invention relates to a duocarmycin derivative-comprising ADC for use in the treatment of a tumor in a human, wherein the duocarmycin derivative-comprising ADC is administered in combination with thiosulfate. An ADC that is suitable for the combined use of the present invention comprises a duocarmycin derivative disclosed in WO2010/062171 as defined below. Such ADC is generically disclosed in WO2010/062171 and WO2011/133039 and can be described by the formula

Ab-(L-D)m,

wherein Ab is an antibody or an antigen-binding fragment of an antibody, L-D is a duocarmycin derivative linker-drug and m represents an average DAR of from 1 to 12.

WO2010/062171 discloses a series of analogues of the DNA-alkylating agent CC-1065. The chemical synthesis of a number of these drugs is described in Examples 1-22 of WO2010/062171.

The duocarmycin derivatives as disclosed in WO2010/062171 consist of a DNA-binding (DB) moiety and a DNA-alkylating (DA) moiety as depicted in formula (IV)

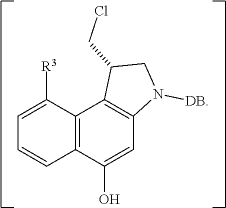

The DB moiety is selected from

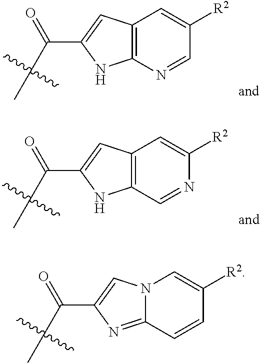

R² and R³ are independently selected from H, OH, SH, NH2, N3, N02, NO, CF3, CN, C(O)NH2, C(O)H, C(O)OH, halogen, RV, SR^{a}, S(O)Ra, S(O)2Ra, S(O)ORa, S(O)2ORa, OS(O)Ra, OS(O)2Ra, OS(O)ORa, OS(O)2ORa, OR^{a}, NHRa, N(Ra)R^{b}, *N(Ra)(R^{b})Rc, P(O)(ORa)(OR^{b}), OP(O)(ORaXOR^{b}), SiRaR^{b}RC, C(O)Ra, C(O)ORa, C(O)N(Ra)R^{b}, OC(O)Ra, OC(O)ORa, OC(O)N(Ra)R^{b}, N(Ra)C(O)R^{b}, N(Ra)C(O)OR^{b}, N(Ra)C(O)N(R^{b})RC, and a water-soluble group, wherein R^{a}, R^{b}, and RC are independently selected from H and optionally substituted (CH2CH2O)aaCH2CH2XlR, CI-I5 alkyl, CI-I5 heteroalkyl, C3-I5 cycloalkyl, CI-I5 heterocycloalkyl, C5-15 aryl, or Ci-is heteroaryl, wherein aa is selected from 1 to 1000, X¹ is selected from 0, S, and NR^{b}I, and R^{b}i and R^{s1} are independently selected from H and C1-3 alkyl, one or more of the optional substituents in R^{a}, R^{b}, and/or RC optionally being a water-soluble group, two or more of R^{a}, R^{b}, and RC optionally being joined by one or more bonds to form one or more optionally substituted carbocycles and/or heterocycles.

The term “water-soluble group” refers to a functional group that is well solvated in aqueous environments and that imparts improved water solubility to the compound to which it is attached. Examples of water-soluble groups include, but are not limited to, polyalcohols, straight chain or cyclic saccharides, primary, secondary, tertiary, or quaternary amines and polyamines, sulfate groups, sulfonate groups, sulfinate groups, carboxylate groups, phosphate groups, phosphonate groups, phosphinate groups, ascorbate groups, glycols, including polyethylene glycols, and polyethers. Preferred water-soluble groups are primary, secondary, tertiary, and quaternary amines, carboxylates, phosphonates, phosphates, sulfonates, sulfates, —(CH2CH₂O)_{yy}CH2CH2X²RY^{Y}, -(CH2CH₂O)_{yy}CH2CH2X²-, -X²(CH2CH₂O)_{yy}CH2CH2-, glycol, oligoethylene glycol, and polyethylene glycol, wherein yy is selected from 1 to 1000, X² is selected from 0, S, and NR”, and R” and RY are independently selected from H and CI-3 alkyl.

The term “substituted”, when used as an adjective to “alkyl”, “heteroalkyl”, “cycloalkyl”, “heterocycloalkyl”, “aryl”, “heteroaryl”, or the like, indicates that said “alkyl”, “heteroalkyl”, “cycloalkyl”, “heterocycloalkyl”, “aryl”, “heteroaryl”, or similar group contains one or more substituents (introduced by substitution for hydrogen). Exemplary substituents include, but are not limited to, OH, ═O, ═S, ═NR^{d}, =N-ORd, SH, NH2, N02, NO, N3, CF3, CN, OCN, SCN, NCO, NCS, C(O)NH2, C(O)H, C(O)OH, halogen, R^{d}, SRd, S(O)Rd, S(O)ORd, S(O)2Rd, S(O)₂ORd, OS(O)Rd, OS(O)ORd, OS(O)2Rd, OS(O)₂ORd, S(O)N(Rd)R^{e}, OS(O)N(R^{d})R^{e}, S(O)₂N(R^{d})R, OS(O)₂N(R^{d})R, OP(O)(OR^{d}XORe), P(O)(ORd)(ORe), ORd, NHRd, N(Rd)Re, *N(Rd)(Re)Rf, Si(Rd)(R)XRf), C(O)Rd, C(O)ORd, C(O)N(R^{d})R_{, OC(O)Rd, OC(O)ORd, OC(O)N(Rd)R*, N(R}^{d})C(O)R_{, N(Rd)C(O)OR*, N(Rd)C(O)N(Re)R}^{f}, a water-soluble group, and the thio derivatives of these substituents, and protonated, charged, and deprotonated forms of any of these substituents, wherein R^{d}, R, and R^{f} are independently selected from H and optionally substituted -(CH2CH₂O)_{yy}CH2CH2X²RY^{Y}, Ci-is alkyl, Ci-is heteroalkyl, C3-is cycloalkyl, Ci-is heterocycloalkyl, C5-15 aryl, or Ci-is heteroaryl, or a combination thereof, wherein yy is selected from 1 to 1000, X² is independently selected from O, S, and NR^{zz}, and R^{zz} and R^{yy} are independently selected from H and C₁₋₃ alkyl, two or more of R^{d}, R^{e}, and R^{f} optionally being joined by one or more bonds to form one or more optionally substituted carbocycles and/or heterocycles. When there is more than one substituent, each substituent is independently selected. Two or more substituents may be connected to each other by replacement of one or more hydrogen atoms on each of the substituents by one or more connecting bonds, which may be single, double, or triple bonds, or, if resonance structures are possible, the bond order of said bonds may be different in two or more of these resonance structures. Two substituents may thus be joined under formation of one or more rings.

When substituents may be “joined by one or more bonds to form one or more optionally substituted carbocycles and/or heterocycles”, this means that the substituents may be connected to each other through replacement of one or more hydrogen atoms on each of the substituents by one or more connecting bonds.

The term “aryl” as used herein refers to a carbocyclic aromatic substituent comprising 5 to 24 ring carbon atoms, which may be charged or uncharged and which may consist of one ring or two or more rings fused together. Examples of aryl groups include, but are not limited to, phenyl, naphthyl, and anthracenyl.

The term “heteroaryl” as used herein refers to a heterocyclic aromatic substituent comprising 1 to 24 ring carbon atoms and at least one ring heteroatom, e.g., oxygen, nitrogen, sulfur, silicon, or phosphorus, wherein nitrogen and sulfur may optionally be oxidized and nitrogen may optionally be quaternized, which may consist of one ring or two or more rings fused together. Heteroatoms may be directly connected to each other. Examples of heteroaryl groups include, but are not limited to, pyridinyl, pyrimidyl, furanyl, pyrrolyl, triazolyl, pyrazolyl, pyrazinyl, oxazolyl, isoxazolyl, thiazolyl, imidazolyl, thienyl, indolyl, benzofuranyl, benzimidazolyl, benzothiazolyl, purinyl, indazolyl, benzotriazolyl, benzisoxazolyl, quinoxalinyl, isoquinolyl, and quinolyl. In one embodiment, a heteroaryl group comprises 1 to 4 heteroatoms. It should be noted that “Ci heteroaryl group” denotes that there is only one carbon present in the ring system of the heteroaromatic group (carbon atoms in optional substituents are thus not counted). An example of such a heteroaromatic group is a tetrazolyl group.

“Aryl” and “heteroaryl” groups also encompass ring systems in which one or more non-aromatic rings are fused to an aryl or heteroaryl ring or ring system.

The term “alkyl” as used herein refers to a straight chain or branched, saturated or unsaturated hydrocarbyl substituent. Examples of alkyl groups include, but are not limited to, methyl, ethyl, propyl, butyl, pentyl, hexyl, octyl, decyl, isopropyl, sec-butyl, isobutyl, tert-butyl, isopentyl, 2-methylbutyl, vinyl, allyl, 1-butenyl, 2-butenyl, isobutylenyl, 1-pentenyl, 2-pentenyl, and 1-butynyl.

The term “heteroalkyl” as used herein refers to a straight chain or branched, saturated or unsaturated hydrocarbyl substituent in which at least one carbon atom is replaced by a heteroatom, e.g., by oxygen, nitrogen, sulfur, silicon, or phosphorus, wherein nitrogen and sulfur may optionally be oxidized and nitrogen may optionally be quaternized. Heteroatoms may be directly connected to each other. Examples include, but are not limited to, methoxy, ethoxy, propoxy, isopropoxy, n-butyloxy, tert-butyloxy, methyloxymethyl, ethyloxymethyl, methyloxyethyl, ethyloxyethyl, methylaminomethyl, dimethylaminomethyl, methylaminoethyl, dimethylaminoethyl, methylthiomethyl, ethylthiomethyl, ethylthioethyl, and methylthioethyl.

The term “cycloalkyl” as used herein refers to a saturated or unsaturated non-aromatic cyclic hydrocarbyl substituent, which may consist of one ring or two or more rings fused together. Examples include, but are not limited to, cyclopropyl, cyclobutyl, cyclopentyl, cyclopentenyl, cyclopentadienyl, cyclohexyl, cyclohexenyl, 1,3-cyclohexadienyl, decalinyl, and 1,4-cyclohexadienyl.

The term “heterocycloalkyl” as used herein refers to a saturated or unsaturated non-aromatic cyclic hydrocarbyl substituent, which may consist of one ring or two or more rings fused together, wherein at least one carbon in one of the rings is replaced by a heteroatom, e.g., by oxygen, nitrogen, sulfur, silicon, or phosphorus, wherein nitrogen and sulfur may optionally be oxidized and nitrogen may optionally be quaternized. Heteroatoms may be directly connected to each other. Examples include, but are not limited to, tetrahydrofuranyl, pyrrolidinyl, piperidinyl, 1,4-dioxanyl, decahydroquinolinyl, piperazinyl, oxazolidinyl, and morpholinyl. It should be noted that “Ci heterocycloalkyl group” denotes that there is only one carbon present in the ring system of the heterocycloalkane (carbon atoms in optional substituents are thus not counted). An example of such a group is a dioxiranyl group.

The term “acyl” as used herein refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₈ acyl group include acetyl-, benzoyl-, nicotinoyl-, propionyl-, isobutyryl-, oxalyl-, and the like.

The number of carbon atoms that an “alkyl”, “heteroalkyl”, “cycloalkyl”, “heterocycloalkyl”, “aryl”, “heteroaryl”, “acyl”, and the like, may contain is indicated by a designation preceding said terms, i.e., C-io alkyl means that said alkyl may contain from one to ten carbons (carbon atoms in optional substituents attached to this alkyl are not counted).

The term “carbocycle” herein refers to a saturated or unsaturated cycloalkane or arene moiety, wherein the terms “cycloalkane” and “arene” are defined as parent moieties of the “cycloalkyl” and “aryl” substituents, respectively, as defined hereinabove.

The term “heterocycle” herein refers to a saturated or unsaturated heterocycloalkane or heteroarene moiety, wherein the terms “heterocycloalkane” and “heteroarene” are defined as parent moieties of the “heterocycloalkyl” and “heteroaryl” substituents, respectively, as defined hereinabove.

The extension “-ylene” as opposed to “-yl” in for example “alkylene” as opposed to “alkyl” indicates that said for example “alkylene” is a divalent (or multivalent) moiety connected to one or more other moieties via at least one or more double bonds or two or more single bonds, as opposed to being a monovalent group connected to one moiety via one single bond in said for example “alkyl”. The term “alkylene” therefore refers to a straight chain or branched, saturated or unsaturated hydrocarbylene moiety; the term “heteroalkylene” as used herein refers to a straight chain or branched, saturated or unsaturated hydrocarbylene moiety in which at least one carbon is replaced by a heteroatom; the term “arylene” as used herein refers to a carbocyclic aromatic moiety, which may consist of one ring or two or more rings fused together; the term “heteroarylene” as used herein refers to a carbocyclic aromatic moiety, which may consist of one ring or two or more rings fused together, wherein at least one carbon in one of the rings is replaced by a heteroatom; the term “cycloalkylene” as used herein refers to a saturated or unsaturated non-aromatic cyclic hydrocarbylene moiety, which may consist of one ring or two or more rings fused together; the term “heterocycloalkylene” as used herein refers to a saturated or unsaturated non-aromatic cyclic hydrocarbylene moiety, which may consist of one ring or two or more rings fused together, wherein at least one carbon in one of the rings is replaced by a heteroatom. Exemplary divalent moieties include those examples given for the monovalent groups hereinabove in which one hydrogen atom is removed.

The prefix “poly” in “polyalkylene”, “polyheteroalkylene”, “polyarylene”, “polyheteroarylene”, “polycycloalkylene”, “polyheterocycloalkylene”, and the like, indicates that two or more of such “-ylene” moieties, e.g., alkylene moieties, are joined together to form a branched or unbranched multivalent moiety containing two or more attachment sites for adjacent moieties. Similarly, the prefix “oligo” in for example oligoethylene glycol indicates that two or more ethylene glycol moieties are joined together to form a branched or unbranched multivalent moiety. The difference between the prefixes “oligo” and “poly” is that the prefix “oligo” is most frequently used to denote a relatively small number of repeating units, while the prefix “poly” usually refers to a relatively large number of repeating units.

Preferably, the duocarmycin derivative of formula (IV) is

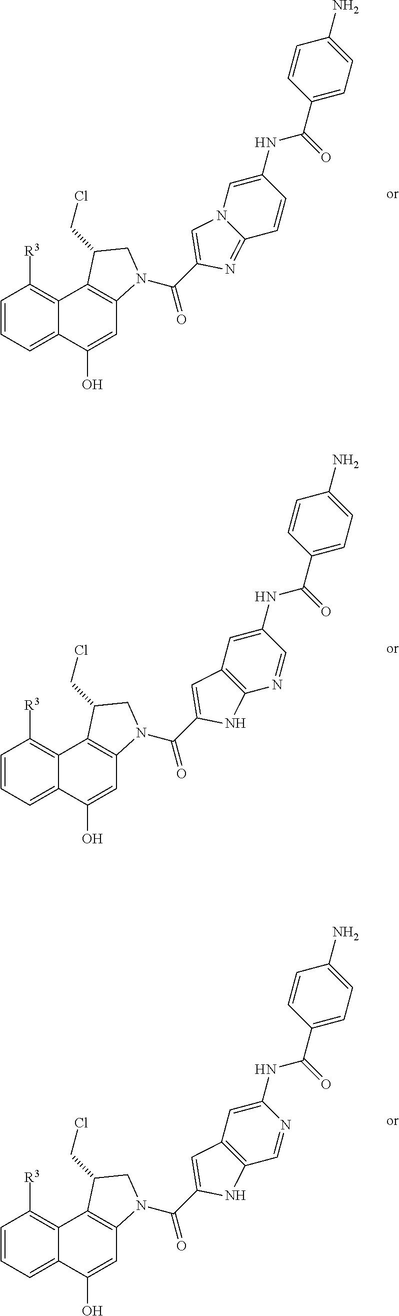

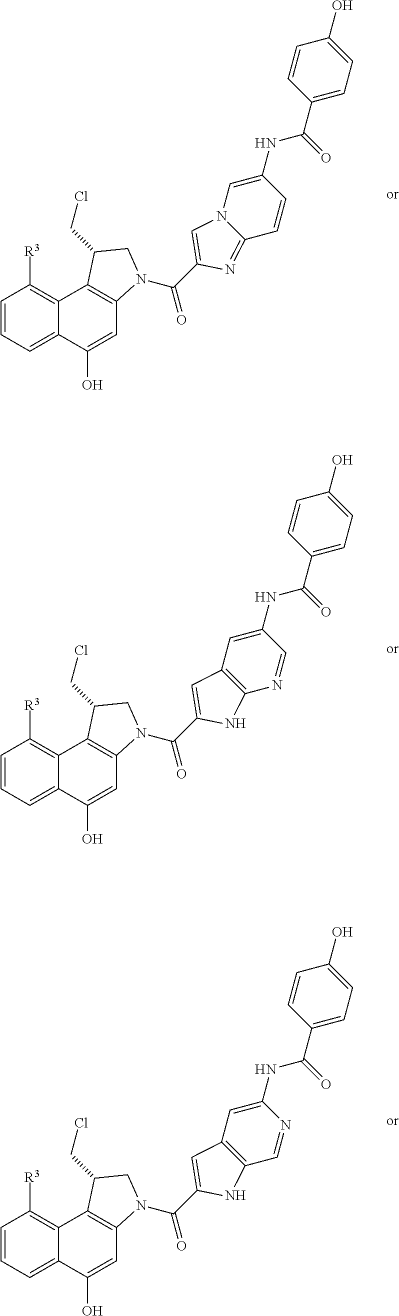

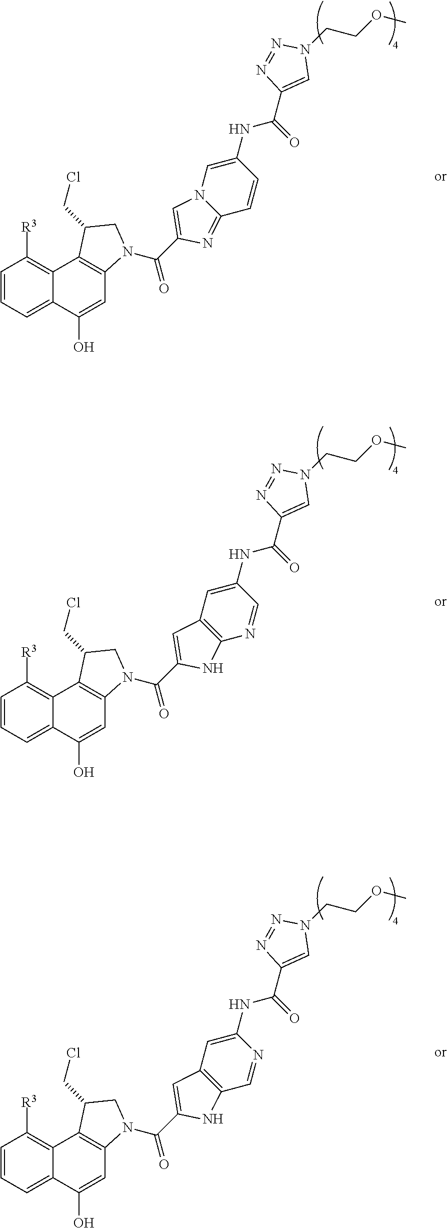

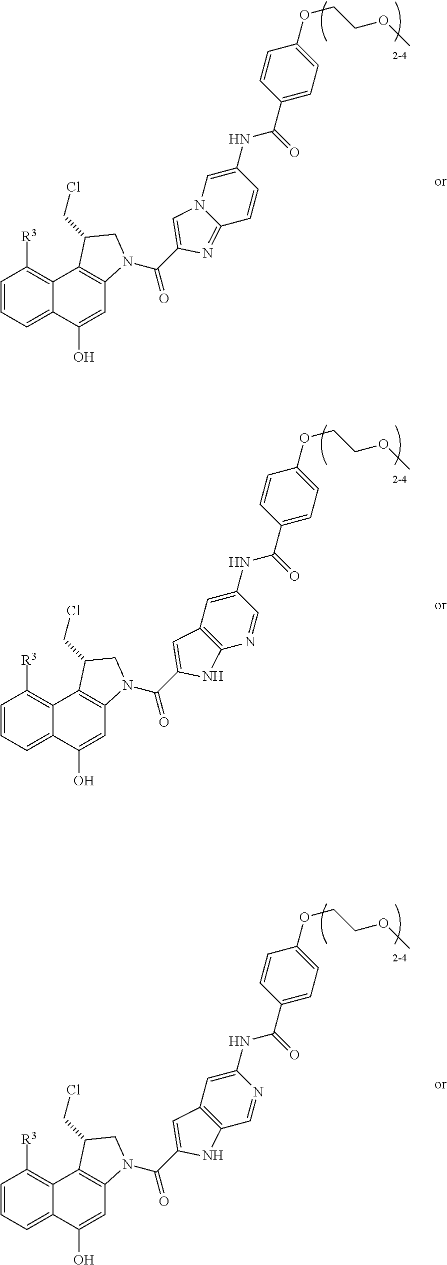

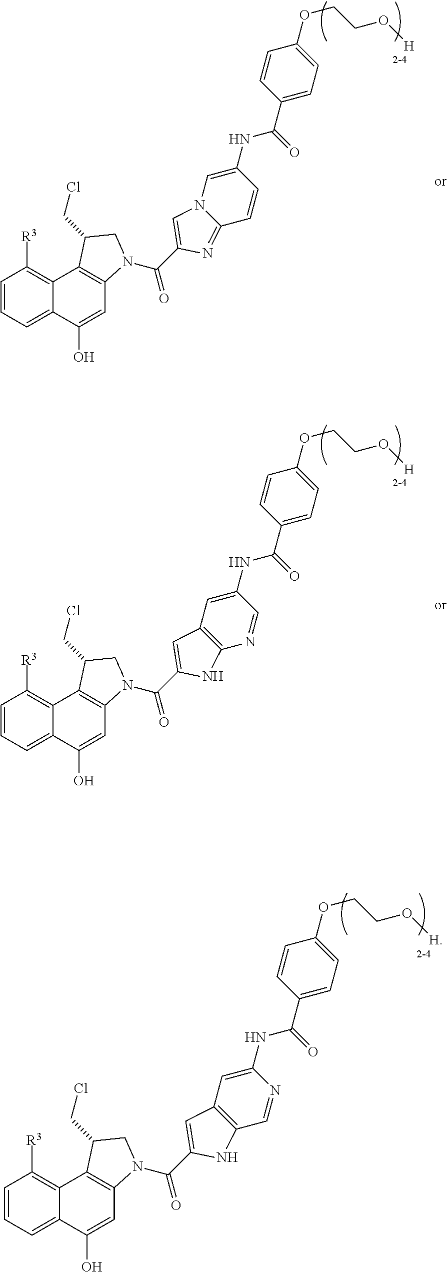

More preferably, R³ is selected from H, methyl and methoxy. Even more preferably, R³ is methyl.

More preferably, the duocarmycin derivative of formula (IV) is

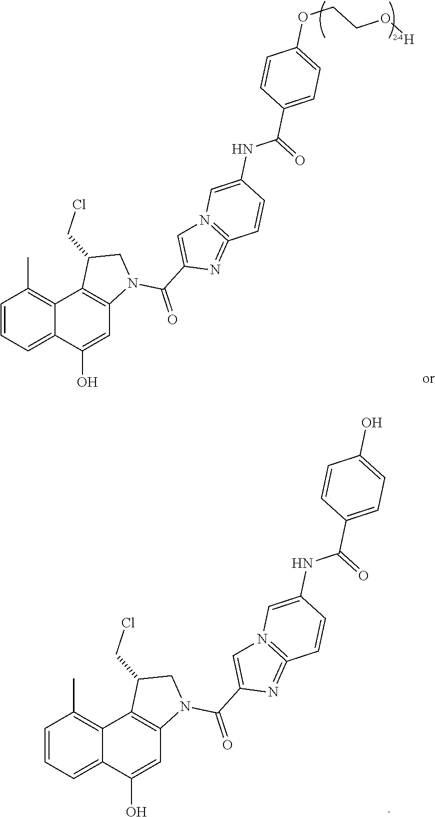

Even more preferably, the duocarmycin derivative of formula (IV) is

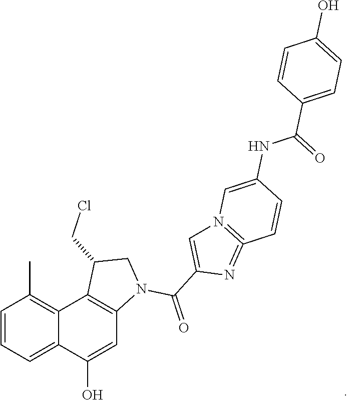

After the observation of ocular toxicity and local toxicity due to extravasation after intravenous administration of trastuzumab duocarmazine of formula (III) in Phase I clinical trials, the present inventors aimed to find a way to prevent and/or reduce these toxic effects, preferably locally at the site of the observed toxicity.

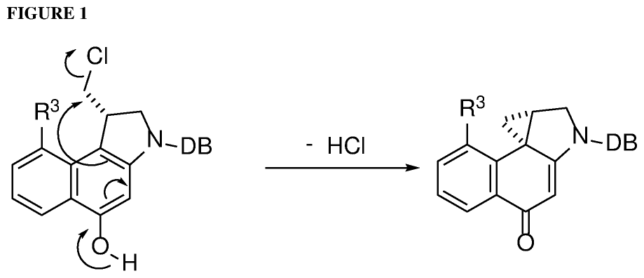

It is believed that a duocarmycin derivative of formula (IV) is converted to an active cyclopropyl-containing compound in vivo with concomitant elimination of HCl, as schematically illustrated in FIG. 1 (Elgersma et al., Molecular Pharmaceutics, 2015, 12(6), 1813-1835). The resulting cyclopropyl-containing compound is the active duocarmycin-derivative drug that exerts the target-specific therapeutic, as well as the unwanted non-target tissue toxicity.

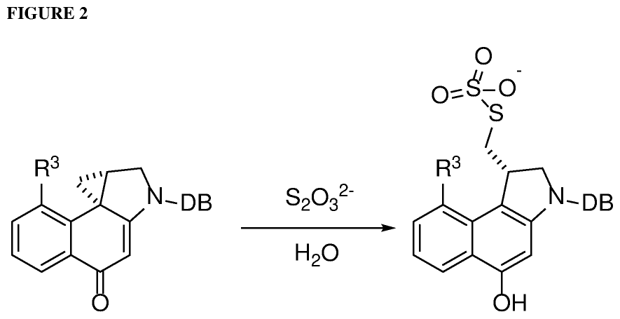

As shown in Example 1, the present invention demonstrates that sodium thiosulfate was able to detoxify the active duocarmycin-derivative drug (i.e., SYD986) released by trastuzumab duocarmazine, while N-acetylcysteine, cysteamine hydrochloride, the sodium salt of 2-mercaptoethanesulfonic acid (MESNA) and L-glutathione were not. Without wishing to be bound by any theory, it is believed that sodium thiosulfate was able to detoxify the active duocarmycin-derivative drug through binding to the cyclopropyl structure, as shown in FIG. 2.

The linker moiety (-L-) of the formula Ab-(L-D)m can be any known or suitable moiety for attaching the drug, in the context of the present invention a duocarmycin derivative of formula (IV), to the antibody or antigen-binding fragment. The linker may be linear or non-linear, as disclosed in e.g., WO2018/069375. Such linker may be cleavable or non-cleavable. Generally, the linker is cleavable under certain conditions, so as to release the drug from the antibody as is known in the art, e.g, a conditionally cleavable or conditionally transformable moiety, which can be cleaved or transformed by a chemical, photochemical, physical, biological, or enzymatic process.

To be able to conjugate a linker or linker-drug moiety to the Ab, the end of the linker that will be (covalently) bonded to the Ab typically contains a functional group that can react with a natural or non-natural amino acid of the Ab under relatively mild conditions. This functional group is referred to herein as a reactive moiety (RM). Examples of reactive moieties include, but are not limited to, carbamoyl halide, acyl halide, active ester, anhydride, a-halo acetyl, a-halo acetamide, maleimide, isocyanate, isothiocyanate, disulfide, thiol, hydrazine, hydrazide, sulfonyl chloride, aldehyde, methyl ketone, vinyl sulfone, halo methyl, and methyl sulfonate.

In a preferred embodiment of the present invention RM is

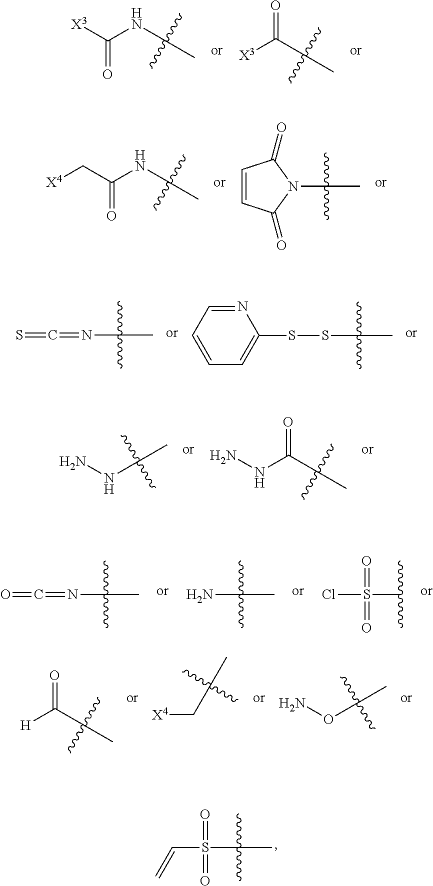

wherein
X³ is selected from —Cl, —Br, —I, —F, —OH, -O-N-succinimide, -O-(4-nitrophenyl),
—O—pentafluorophenyl, -O-tetrafluorophenyl, —O—C(O)—R⁴, and —O—C(O)—OR⁴;
X⁴ is selected from —Cl, —Br, —I, -O-mesyl, -O-triflyl, and -O-tosyl; and
R⁴ is branched or unbranched C₁--C₁₀ Calkyl or aryl.

In a preferred embodiment, the present invention relates to an ADC for use in the treatment of a tumor in a mammal, preferably a human, wherein the ADC is administered in combination with thiosulfate and wherein the ADC is a compound of formula (I)

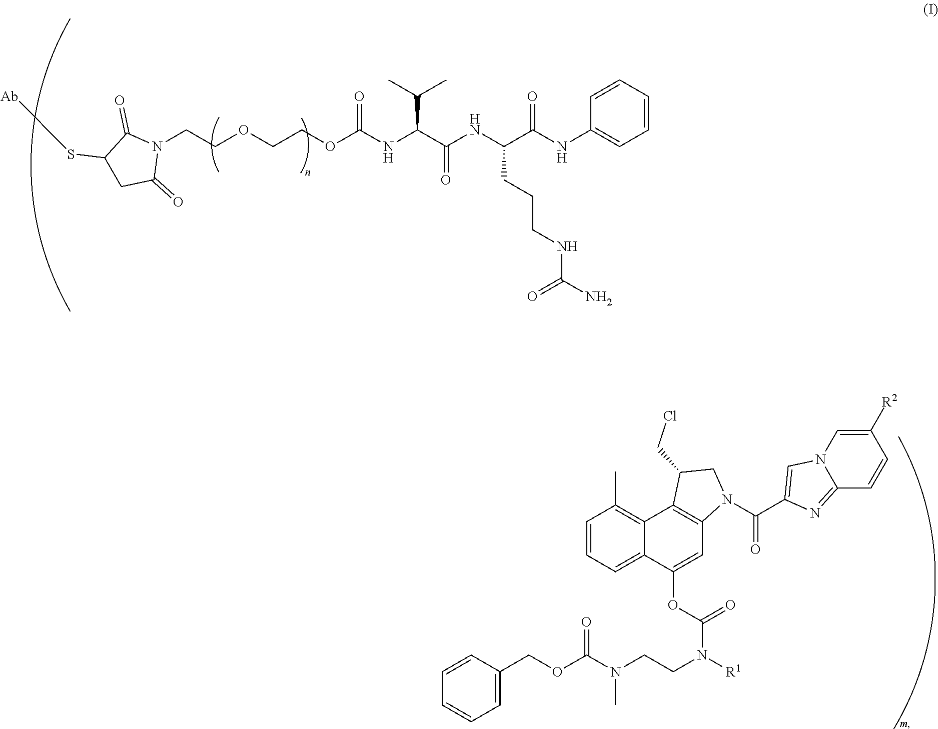

wherein
Ab is an antibody or an antigen-binding fragment of an antibody; ^{PGP}-3⁴,Ci
n is0, 1,2or3;
m represents an average DAR of from 1 to 6;
R¹ is selected from the group consisting of

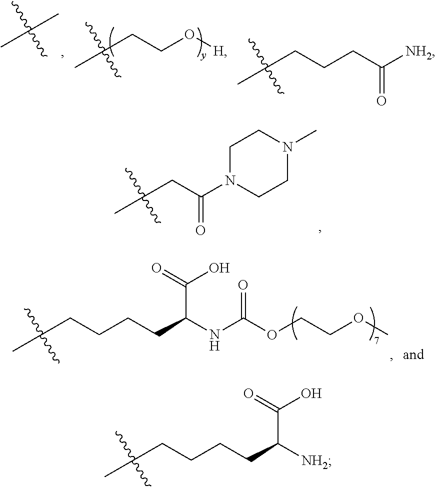

y is an integer of from 1-16; and
R² is selected from the group consisting of

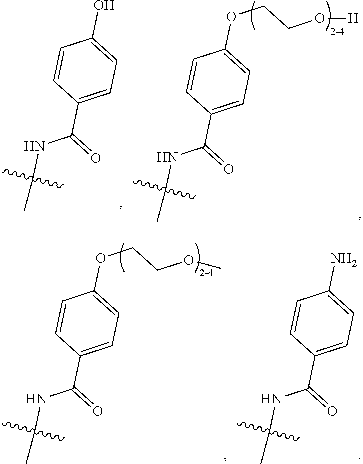

In a preferred embodiment, n is 0 or 1;
m represents an average DAR of from 1 to 4; R¹ is

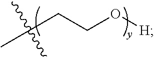

y is an integer of from 1-4; and R²is selected from the group consisting of

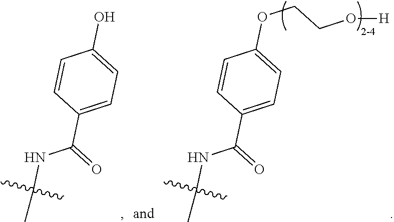

In a more preferred embodiment, the ADC is a compound of formula (II)

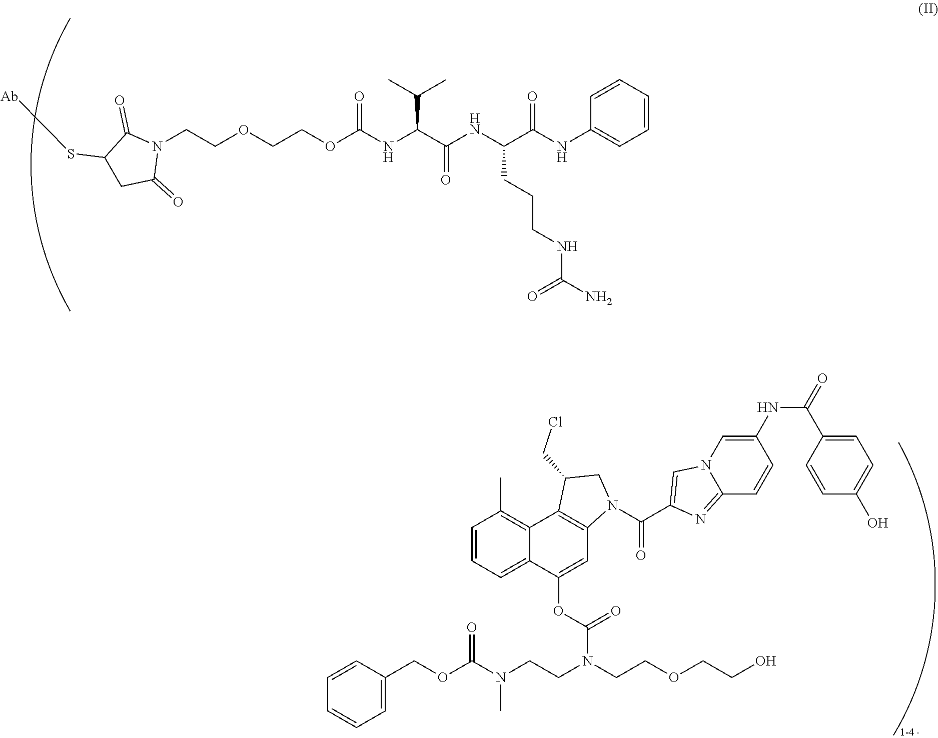

In the context of the present invention, Ab in the ADC formulae (I) and (II) can be any P-39,c1 antibody or an antigen-binding fragment thereof, preferably a monoclonal antibody (mAb) or an antigen-binding fragment thereof.

The term “antibody” as used herein preferably refers to an antibody comprising two heavy chains and two light chains. Generally, the antibody or any antigen-binding fragment thereof is one that has a therapeutic activity, but such independent efficacy is not necessarily required, as is known in the art of ADCs. The antibodies to be used in accordance with the invention may be of any isotype such as IgA, IgE, IgG, or IgM antibodies. Preferably, the antibody is an IgG antibody, more preferably an IgGi or IgG2 antibody. The antibodies may be chimeric, humanized or human. Preferably, the antibodies are humanized or human. Even more preferably, the antibody is a humanized or human IgG antibody, more preferably a humanized or human IgGi mAb, most preferably a humanized IgGi mAb. The antibody may have κ(kappa) or λ(lambda) light chains, preferably κ(kappa) light chains, i.e., a humanized or human IgG_{1-κ} antibody.

The term “antigen-binding fragment” as used herein includes a Fab, Fab′, F(ab′)2, Fv, scFv or reduced IgG (rIgG) fragment, a single chain (sc) antibody, a single domain (sd) antibody, a diabody, or a minibody. “Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable regions (VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. Exemplary methods for humanization of non-human antibodies are the method of Winter and co-workers (Jones et al., Nature 1986, 321, 522-525; Riechmann et al., Nature 1988, 332, 323-327; Verhoeyen et al., Science 1988, 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al., Nature 1986, 321, 522-525; Riechmann et al., Nature 1988, 332, 323-327; and Presta, Curr. Op. Struct. Biol. 1992, 2, 593-596. See also the following review articles and references cited therein: Vaswani and Hamilton, Ann. Allergy, Asthma and Immunol. 1998, 1, 105-115; Harris, Biochem. Soc. Transactions 1995, 23, 1035-1038; and Hurle and Gross, Curr. Op. Biotech. (1994), 5, 428-433.

The CDRs may be determined using the approach of Kabat (in Kabat et al., Sequences of Proteins of Immunological Interest, 5th Ed. Public Health Service, National Institutes of Health, Bethesda, Md., NIH publication no. 91-3242, pp. 662, 680, 689 (1991)), Chothia (Chothia et al., Nature 1989, 342, 877-883) or IMGT (Lefranc, The Immunologist 1999, 7, 132-136).

Typically, the antibody is a monospecific (i.e., specific for one antigen; such antigen may be common between species or have similar amino acid sequences between species) or bispecific (i.e., specific for two different antigens of a species) antibody comprising at least one HC and LC variable region binding to a tumor associated antigen (TAA). Preferably, the TAA is a membrane bound TAA, which may be internalizing or not internalizing, preferably internalizing.

In one particular embodiment, the TAA is selected from the group consisting of: annexin Al, B7H3, B7H4, CA6, CA9, CA15-3, CA19-9, CA27-29, CA125, CA242 (cancer antigen 242), CCR2, CCR5, CD2, CD19, CD20, CD22, CD30 (tumor necrosis factor 8), CD33, CD37, CD38 (cyclic ADP ribose hydrolase), CD40, CD44, CD47 (integrin associated protein), CD56 (neural cell adhesion molecule), CD70, CD74, CD79, CD 115 (colony stimulating factor 1 receptor), CD123 (interleukin-3 receptor), CD138 (Syndecan 1), CD203c (ENPP3), CD303, CD333, CDCP1, CEA, CEACAM, CLCA-1 (C-type lectin-like molecule-1), CLL-1, c-MET (hepatocyte growth factor receptor), Cripto, DLL3, EGFL, EGFR, EPCAM, EPh (e.g., EphA2 or EPhB3), ETBR (endothelin type B receptor), FAP, FcRL5 (Fc receptor-like protein 5, CD307), FGFR (e.g., FGFR3), FOLR1 (folate receptor alpha), GCC (guanylyl cyclase C), GPNMB, HER2, p95HER2, HMW-MAA (high molecular weight melanoma-associated antigen), integrin a (e.g., avP3 and avP5), IGF1R, TM4SF1(or L6), Lewis A like carbohydrate, Lewis X, Lewis Y (CD174), LIV1, mesothelin (MSLN), MN (CA9), MUC1, MUC16, NaPi2b, Nectin-4, PD-1, PD-L1, PSMA, PTK7, SLC44A4, STEAP-1, 5T4 (or TPBG, trophoblast glycoprotein), TF (tissue factor, thromboplastin, CD142), TF-Ag, Tag72, TNFR, TROP2 (tumor-associated calcium signal transducer 2), VEGFR and VLA.

Examples of suitable antibodies include blinatumomab (CD19), epratuzumab (CD22), iratumumab and brentuximab (CD30), vadastuximab (CD33), tetulumab (CD37), isatuximab (CD38), bivatuzumab (CD44), lorvotuzumab (CD56), vorsetuzumab (CD70), milatuzumab (CD74), polatuzumab (CD79), rovalpituzumab (DLL3), futuximab (EGFR), oportuzumab (EPCAM), farletuzumab (FOLR1), glembatumumab (GPNMB), trastuzumab, pertuzumab and margetuximab (HER2), etaracizumab (integrin), anetumab (mesothelin), pankomab (MUCi), enfortumab (Nectin-4), and H8, Al, and A3 (5T4).

In a more particular embodiment, the present invention relates to an ADC compound as described hereinabove wherein the antibody comprised in the ADC is an anti-annexin Al antibody, an anti-B7H3 antibody, an anti-CD115 antibody, an anti-CD123 antibody, an anti-CLL-1 antibody, an anti-c-MET antibody, an anti-HER2 antibody, an anti-MUCi antibody, an anti-PSMA antibody, an anti-5T4 antibody or an anti-TF antibody, preferably an ADC compound in accordance with formula (I) or (II).

In a preferred embodiment, Ab in the compound of formula (I) or (II) is the anti-HER2 antibody trastuzumab.

In a more preferred embodiment, the ADC is a compound of formula (III)

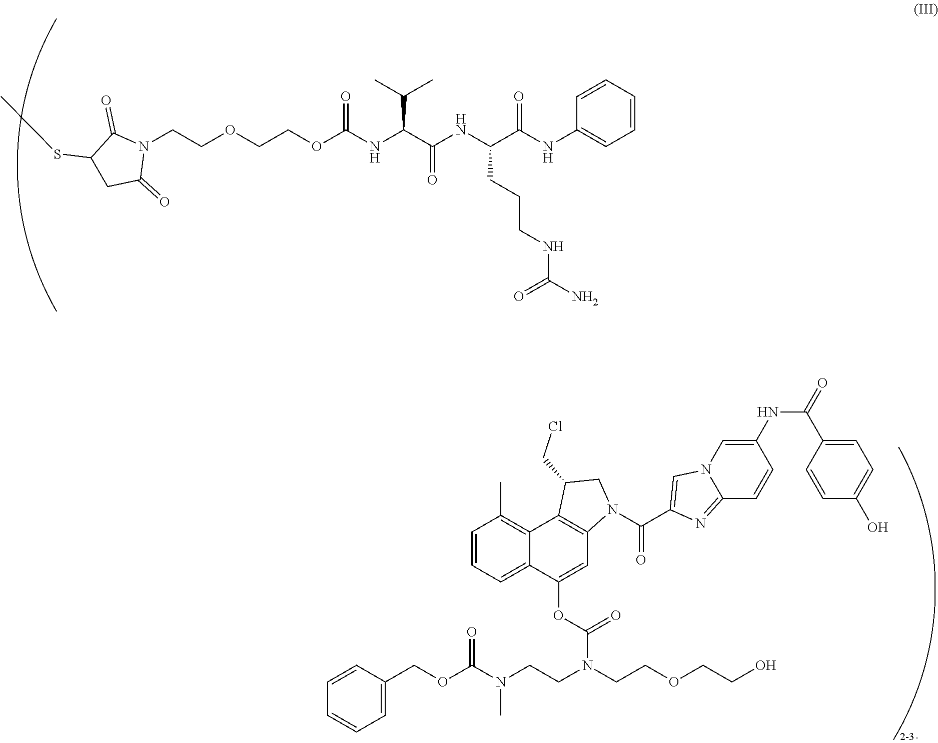 [SMILES: CSC1CC(=O)N(CCOCCOC(=O)N[C@H](C(=O)N[C@@H](CCCNC(N)=O)C(=O)Nc2ccccc2)C(C)C)C1=O ; Cc1cccc2c(OC(=O)N(CCOCCO)CCN(C)C(=O)OCc3ccccc3)cc3c(c12)[C@H](CCl)CN3C(=O)c1cn2cc(NC(=O)c3ccc(O)cc3)ccc2n1]

The antibody or antigen-binding fragment thereof, if applicable, may comprise (1) a constant region that is engineered, i.e., one or more mutations may have been introduced to e.g., increase half-life, provide a site of attachment for the linker-drug and/or increase or decrease effector function; or (2) a variable region that is engineered, i.e., one or more mutations may have been introduced to provide a site of attachment for the linker-drug. Antibodies or antigen-binding fragments thereof may be produced recombinantly, synthetically, or by other known suitable methods.

ADCs for use in the present invention may be wild-type or site-specific, and can be produced by any method known in the art as exemplified below.

Wild-type ADCs may be produced by conjugating a linker-drug to the antibody or antigen-binding fragment thereof through e.g., the lysine E-amino groups of the antibody, preferably using a linker-drug comprising an amine-reactive group such as an activated ester; contacting of the activated ester with the antibody or antigen-binding fragment thereof will yield the ADC. Alternatively, wild-type ADCs can be produced by conjugating the linker-drug through the free thiols of the side chains of cysteines generated through reduction of interchain disulfide bonds, using methods and conditions known in the art, see e.g., Doronina et al., Bioconjugate Chem. 2006, 17, 114-124. The manufacturing process involves partial reduction of the solvent-exposed interchain disulfides followed by modification of the resulting thiols with Michael acceptor-containing linker-drugs such as maleimide-containing linker-drugs, alfa-haloacetic amides or esters. The cysteine attachment strategy results in maximally two linker-drugs per reduced disulfide. Most human IgG molecules have four solvent-exposed disulfide bonds, and so a range of integers of from zero to eight linker-drugs per antibody is possible. The exact number of linker-drugs per antibody is determined by the extent of disulfide reduction and the number of molar equivalents of linker-drug used in the ensuing conjugation reaction. Full reduction of all four disulfide bonds gives a homogeneous construct with eight linker-drugs per antibody, while a partial reduction typically results in a heterogeneous mixture with zero, two, four, six, or eight linker-drugs per antibody.

Site-specific ADCs are preferably produced by conjugating the linker-drug to the antibody or antigen-binding fragment thereof through the side chains of engineered cysteine residues in suitable positions of the mutated antibody or antigen-binding fragment thereof. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Under optimal conditions, two linker-drugs per antibody or antigen-binding fragment thereof (i.e., drug-to-antibody ratio, DAR, is 2) will be attached (if one cysteine is engineered into the HC or LC of the mAb or fragment). Suitable methods for site-specifically conjugating linker-drugs can for example be found in WO2015/177360 which describes the process of reduction and re-oxidation, WO2017/137628 which describes a method using mild reducing agents and WO2018/215427 which describes a method for conjugating both the reduced interchain cysteines as well as the uncapped engineered cysteines.

A tumor which is treated in the context of the present invention, preferably is a tumor expressing the antigen to which the ADC is directed. Such tumors may be human solid tumors or hematological malignancies. Examples of tumors that may be treated with an ADC as defined above may include, but are not limited to breast cancer, brain cancer (e.g., glioblastoma), head and neck cancer, thyroid cancer, adrenal cancer (e.g., neuroblastoma), bone cancer (e.g., osteosarcoma), ocular cancer, esophageal cancer, gastric cancer, small intestine cancer, colorectal cancer, urothelial cancer (e.g., bladder or renal cancer), ovarian cancer, uterine cancer, vaginal and cervical cancer, lung cancer (especially non-small cell lung cancer (NSCLC) and small-cell lung cancer (SCLC)), melanoma, mesothelioma (especially malignant pleural mesothelioma), liver cancer (e.g., hepatocellular carcinoma), pancreatic cancer, skin cancer, testicular cancer, prostate cancer, acute myeloid leukemia (AML), chronic myeloid leukemia (CML), chronic lymphatic leukemia (CLL), acute lymphoblastic leukemia (ALL), non-Hodgkin's lymphoma (NHL) (including follicular lymphoma (FL) and diffuse large B-cell lymphoma (DLBCL)), and multiple myeloma (MM).

In the context of the present invention, “thiosulfate” may be in any form; i.e., it may be a thiosulfate acid, e.g., thiosulfuric acid, or a thiosulfate salt, e.g., ammonium thiosulfate, calcium thiosulfate, potassium thiosulfate or sodium thiosulfate (STS), each containing the anionic moiety S₂O₃²⁻- and a suitable countercation. Preferably, the thiosulfate is a thiosulfate salt, more preferably STS.

STS (also known as sodium hyposulfite) is an inorganic compound with the formula Na₂S₂O₃·xH₂O. Typically, it is available as the pentahydrate, Na₂S₂O₃·5H₂O. Since it is employed as a food preservative, the general population is widely exposed to this compound, which is considered non-toxic (McGeer et al., Journal of Neurology and Neuromedicine 2016, 1, 28-30). STS came into medical use as an antidote for cyanide poisoning in the 1930s. Other medical uses include treatment of ringworm, pityriasis versicolor and other fungal infections of the skin, and reduction of side effects from cisplatin, such as nephro- and ototoxicity, and local toxicity caused by extravasation. STS is on the World Health Organization's List of Essential Medicines, the most effective and safe medicines needed in a health system.

For cyanide poisoning, STS is given to patients by intravenous injection in dosages up to 12.5 g. When used to reduce the nephrotoxicity of cisplatin, the first dose of 4 g/m² body surface area STS is given intravenously to patients just before the cisplatin, followed by a second dose of usually 12 g/m² at the same time as the cisplatin. In case of extravasation of cisplatin, 2 ml of a 0.167 M STS solution is injected intravenously for every 100 ml of cisplatin, which is followed by 0.1 ml subcutaneous injections in a clockwise pattern around the extravasation area up to 1 ml. This procedure is repeated several times within 3-4 hours of the extravasation incident. For the treatment of cisplatin-related ototoxicity, STS is applied as a 0.1 M solution in hyaluronan gel in the ear. In the treatment of pityriasis versicolor, STS is applied dermally twice daily for four weeks as a 15% lotion formulation.

Intravenous STS (37.5-75.0 g/week) has also been reported to improve skin lesions in dialysis patients affected by calciphylaxis. In addition, despite the low oral uptake of STS, several studies describe the successful use of up to 7.5 g/week oral STS in such patients (Musso et al., Saudi Journal of Kidney Diseases and Transplantation 2008, 19, 820-821; Shetty and Klein, Advances in Peritoneal Dialysis 2016, 32, 51-55).

STS is also often used as an excipient in pharmaceutical formulations, including ophthalmic formulations.

ADCs are typically administered via intravenous infusion. Administration of the ADC “in combination with” thiosulfate or vice versa, is herein understood to mean that the ADC and the thiosulfate are administered as components of the same treatment regime, wherein the thiosulfate is administered to prevent or to treat unwanted non-target tissue toxicity. However, it is not required that the thiosulfate and the ADC are comprised in a single pharmaceutical formulation. In a preferred embodiment, the ADC and the thiosulfate are administered simultaneously, separately or sequentially. Determination of the appropriate dose and dosage form depend inter alia on the unwanted non-target tissue toxicity to be prevented or treated and is made by the clinician, e.g., using parameters or factors known or suspected in the art to affect treatment or predicted to affect treatment. A treatment schedule may for instance include a first administration of thiosulfate from about three weeks before to about 1 hour after the first administration of the ADC and subsequent administrations of the thiosulfate at regular intervals until up to three months after the last administration of the ADC. Such regular intervals may for example include, but are not limited to thrice daily, twice daily, daily, weekly, biweekly.

In a further embodiment, the thiosulfate is administered by inhalation or via intravenous, oral, dermal, subcutaneous or ocular route.

In a preferred embodiment, the thiosulfate is administered via intravenous route. In a more preferred embodiment, the thiosulfate is administered via intravenous route followed by administration via subcutaneous route as may be applied in the treatment of toxicity caused by extravasation of the ADC. Typically, following extravasation, an effective amount of a thiosulfate solution is injected intravenously, followed by several subcutaneous injections of an effective amount of the thiosulfate solution around the extravasation area. Generally, up to 10 ml of a 0.05-0.5 M thiosulfate solution is injected intravenously for every 100 ml of ADC, which is followed by 0.1 ml subcutaneous injections in a clockwise pattern around the extravasation area up to 1 ml. This procedure is repeated several times within 3-12 hours of the extravasation incident, preferably within 3-8 hours, more preferably within 3-6 hours, most preferably within 3-4 hours.

In another preferred embodiment, the thiosulfate is administered via ocular route. Typically, an effective amount of the thiosulfate is administered in eye drops. Generally, 1 or 2 eye drops of a 0.0313-0.5 M thiosulfate solution are administered to each eye from 1 to 24 times a day.

In a further aspect, the present invention provides a composition comprising thiosulfate, preferably wherein the composition is a pharmaceutical composition, more preferably further comprising a pharmaceutically acceptable carrier. Such composition is referred to hereinafter as a composition according to the invention. The composition may for example be a liquid formulation, a lyophilized formulation, or in the form of e.g., a capsule or a tablet.

The preferred form depends on the intended mode of administration and therapeutic application. The pharmaceutical carrier can be any compatible, nontoxic substance suitable to deliver the thiosulfate to a subject. Pharmaceutically acceptable carriers are well known in the art and include, for example, one or more of an aqueous solution such as (sterile) water or physiologically buffered saline or a solvent or vehicle such as glycol, glycerol, hyaluronic acid (or hyaluronan), an oil such as olive oil or an injectable organic ester, alcohol, fat, wax, and an inert solid. A pharmaceutically acceptable carrier may further contain a physiologically acceptable compound that acts for example to stabilize or to increase the absorption of the thiosulfate. Such a physiologically acceptable compound includes, for example, one or more of a carbohydrate, such as glucose, sucrose, or dextran, an antioxidant, such as ascorbic acid or glutathione, a chelating agent, a low molecular weight protein, or another stabilizer or excipient. One skilled in the art knows that the choice of a pharmaceutically acceptable carrier, including a physiologically acceptable compound, depends, for example, on the route of administration of the composition. A pharmaceutical composition of the invention may further comprise one or more of a pharmaceutically acceptable adjuvant, a buffering agent (e.g., citrate, an amino acid such as histidine, or a succinate containing salt in water), a lyoprotectant (e.g., sucrose, trehalose), a tonicity modifier (e.g., a chloride salt such as sodium chloride), a surfactant (e.g., polysorbate), a bulking agent (e.g., mannitol, glycine) and the like.

For oral administration, thiosulfate can be administered in a solid dosage form, such as a capsule, a tablet, and a powder, or in a liquid dosage form, such as an elixir, a syrup, or a suspension. Thiosulfate can for example be encapsulated in a gelatin capsule together with inactive ingredients and powdered carriers, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion, or other known desirable features are red iron oxide, silica gel, sodium lauryl sulfate, titanium dioxide, edible white ink, and the like. Similar diluents can be used to make compressed tablets. Both tablets and capsules can be manufactured as sustained release products to provide for continuous release of thiosulfate over a period of hours. Compressed tablets can be sugar-coated or film-coated to mask any unpleasant taste and protect the tablet from the atmosphere, or enteric-coated for selective disintegration in the gastrointestinal tract. Liquid dosage forms for oral administration can contain coloring and flavoring to increase patient acceptance. Preparations for parenteral administration must be sterile. Sterilization is readily accomplished by filtration through sterile filtration membranes, optionally prior to or following lyophilization and reconstitution. The parenteral route for administration is in accord with known methods, e.g., injection or infusion by intravenous, intraperitoneal, intramuscular, intraarterial, or intralesional routes. The composition may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the active compound, depending on the particular type of compound and its required dosing regimen. Methods for preparing parenterally administrable compositions are well known in the art and described in more detail in various sources, including, for example, Remington's Pharmaceutical Science (17th ed., Mack Publishing, Easton, Pa., 1985).

The composition according to the invention can be an immediate release composition or a composition with delayed, modified or sustained release.

In one embodiment, the present invention relates to a pharmaceutical composition comprising up to 1.0 M STS and hyaluronic acid, wherein the composition is a liquid composition suitable for ocular use. Preferably, the composition comprises up to 0.5 M STS, such as 0.0313, 0.05, 0.063, 0.10, 0.125, 0.20, 0.25, or 0.5 M.

In another embodiment, the present invention relates to a pharmaceutical composition comprising up to 250 mg/ml STS and at least one pharmaceutically acceptable carrier, wherein the composition is a liquid composition suitable for intravenous use. Suitable pharmaceutical carriers are for example water for injections, saline or potassium chloride solution, and sodium hydroxide or boric acid for pH adjustment.

In another embodiment, the present invention relates to a pharmaceutical composition comprising up to 25% STS and at least one pharmaceutically acceptable carrier, wherein the composition is a liquid composition suitable for dermal use. Suitable pharmaceutical carriers are for example water for injections, isopropyl alcohol and propylene glycol.

In a further aspect, the present invention provides a composition comprising thiosulfate for use in a mammal, preferably a human, in the prevention or reduction of non-target tissue toxicity associated with the administration to the mammal of an ADC as defined hereinabove.

In a further aspect, the present invention provides for a use of an ADC as defined hereinabove, in the manufacture of a medicament for use in combination therapy for treatment of a tumor in a mammal, preferably a human, wherein the medicament is administered in combination with thiosulfate as defined hereinabove.

In a further aspect, the present invention provides a product containing an ADC as defined hereinabove and thiosulfate as a combined preparation for the simultaneous, separate or sequential use in the treatment of a tumor in a mammal, preferably a human. A “combined preparation” as used herein defines especially a “kit of parts” in the sense that the combination partners as defined hereinabove can be dosed independently or by use of different fixed combinations with distinguished amounts of the combination partners, i.e., simultaneously, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. The product preferably is a pharmaceutical product. The product can be included in a container, pack, or dispenser optionally together with instructions for administration.

In a further aspect, the present invention provides a method for preventing or reducing non-target tissue toxicity associated with the administration of an ADC as defined hereinabove, comprising administering an effective amount of the ADC in combination with an effective amount of thiosulfate. In an embodiment, the thiosulfate is administered from about three weeks before to about 1 hour after the first administration of the ADC and the administration of thiosulfate is repeated at regular intervals until up to three months after treatment with the ADC has ended, i.e., up to three months after the last administration of the ADC. Such regular intervals may for example include, but are not limited to thrice daily, twice daily, daily, weekly, biweekly. In a preferred embodiment, the thiosulfate is administered after the first administration of the ADC. In another preferred embodiment, the ADC is administered after the first administration of the thiosulfate.

In a further aspect, the present invention relates to a method of treating, preventing or reducing non-target toxicity associated with the administration of an ADC as defined hereinabove, which method comprises administering to a subject in need thereof a therapeutically effective amount of thiosulfate. The term “subject” as used herein refers to all animals classified as mammals and includes, but is not restricted to, primates and humans.

The subject is preferably a human. The expression “therapeutically effective amount” means an amount effective in treating, preventing, or reducing the unwanted toxicity associated with administration of the ADC; said amount can be an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the present specification are hereby incorporated by reference in their entirety.

The following Examples are offered for illustrative purposes only, and are not intended to limit the scope of the present invention in any way.

### Examples

Materials and methods

LC-MS - For LC-MS, 10 μL of sample was injected onto an Acquity UPLC BEH Shield RP18 column (particle size 1.7 pm, 1.0 mm ID ×100 mm, Waters, Cat. no. 1270) at a flow rate of 0.11 ml/min and at a column temperature of 45° C. The elution method is depicted in the table below. The composition of mobile phase A was 0.1% formic acid in Milli-Q water, the mobile phase B was acetonitrile. The run time was 9.0 minutes. A Waters Acquity UPLC system equipped with a MicroTOF Q II mass spectrometer (Bruker) and Empower (UPLC) and Bruker software (MS Q-ToF) was used. UV absorbance was measured at 275-279 nm.

Gradient program

| Time | Mobile phase A | Mobile phase B |
|---|---|---|
| [min] | [%] | [%] |
| 0 | 80 | 20 |
| 4.0 | 37 | 63 |
| 5.0 | 5 | 95 |
| 6.5 | 5 | 95 |
| 7.0 | 80 | 20 |
| 9.0 | 80 | 20 |

### Example 1- Detoxification experiments with thiol compounds

Experiments with cyclopropyl-containing (duocarmycin) compound SYD986

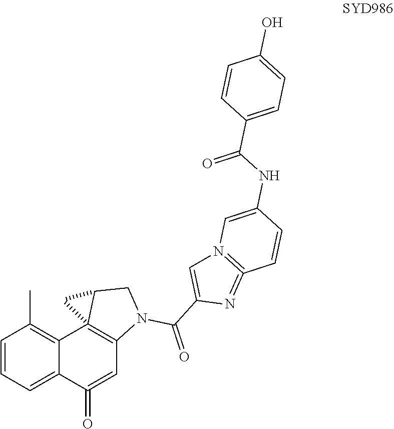

SYD986 was dissolved at a concentration of 5.0 pg/ml (10 pM) in dimethylacetamide (DMA) with 0.1% acetic acid. The thiols, as listed in the table below, were dissolved in acetonitrile/water (1:1).

| | Concentration in |
|---|---|
| Thiol | acetonitrile/water (1:1) (mM) |
| N-Acetyleysteine | 11.1 |
| Cysteamine HCl | 7.54 |
| 2-Mercaptoethanesulfonic | 11.1 |
| acid sodium salt (MESNA) | |
| L-Glutathione | 1 |
| Sodium thiosulfate (STS) | 1 |

The detoxification test solution was prepared by mixing 900 pl of the thiol solution with 100 μl SYD986 solution (SYD986 1 μM; thiol 6.8 mM (cysteamine)/10 mM (other thiols)). LC-MS analysis was performed at the start of the experiment and after various time intervals.

1 μM SYD986 blank solution was prepared by the dilution of 100 μl SYD986 solution (5.0 μg/ml) with 900 μl acetonitrile/water (1:1).

N-Acetylcysteine, glutathione, cysteamine and MESNA did not form a reaction product with SYD986. Instead, slow hydrolysis of SYD986 occurred and a small peak (visible after about 22 hours reaction time) was formed with m/z 509.19; [M+18]⁺corresponding to

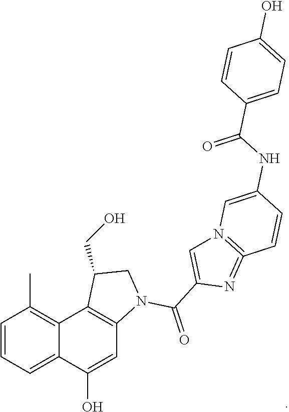

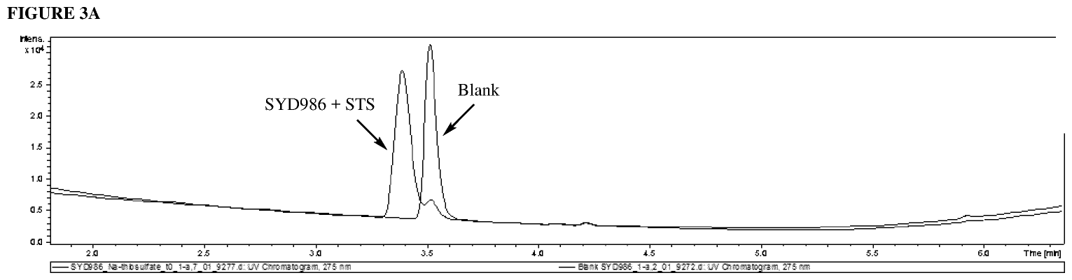

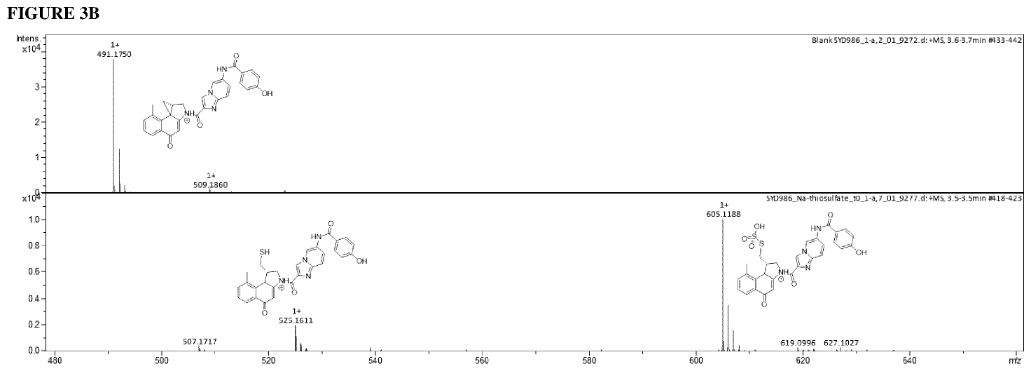

In contrast with the other thiols, STS did react with SYD986, resulting in a reaction product with m/z 605.12 [M+H]*. Moreover, this reaction was relatively fast. FIG. 3A shows a comparison of the UV chromatograms of SYD986 blank solution (1 μM) and SYD986 (1 μM)+STS (10 mM) dissolved in acetonitrile/water (1:1) after a reaction time of about 1.5 hours at 20° C. FIG. 3B shows the MS analysis results of the SYD986 blank (upper panel) and the reaction product of SYD986 and STS (lower panel).

Experiments with quenched linker-drug vc-seco-DUBA

To verify that thiosulfate only reacts with the cyclopropyl-containing compound SYD986 and not with the linker-drug (vc-seco-DUBA) at e.g., the chlorine atom, thiosulfate was added to (N-acetylcysteine-)quenched vc-seco-DUBA. Quenched linker-drug was used instead of vc-seco-DUBA to prevent reaction of thiosulfate with the maleimide moiety of the linker-drug.

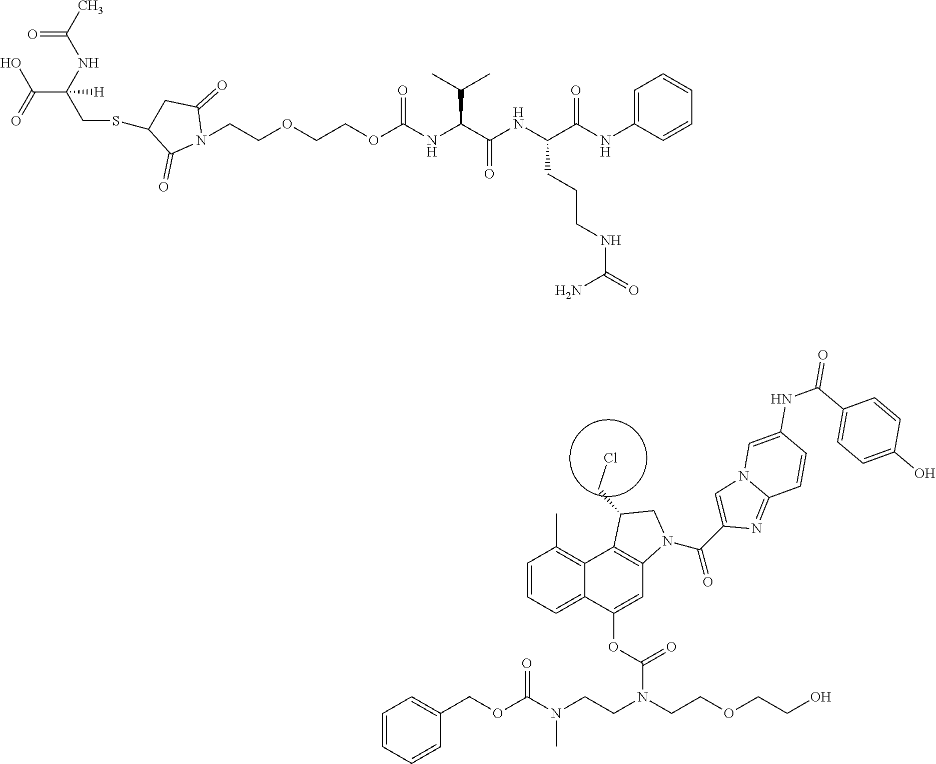 [SMILES: CC(=O)N[C@@H](CSC1CC(=O)N(CCOCCOC(=O)N[C@H](C(=O)N[C@@H](CCCNC(N)=O)C(=O)Nc2ccccc2)C(C)C)C1=O)C(=O)O ; Cc1cccc2c(OC(=O)N(CCOCCO)CCN(C)C(=O)OCc3ccccc3)cc3c(c12)[C@H](CCl)CN3C(=O)c1cn2cc(NC(=O)c3ccc(O)cc3)ccc2n1]

Quenched linker-drug was dissolved and diluted in DMA with 0.1% acetic acid at a concentration of 15.0 μg/ml (10 μM). STS was dissolved in acetonitrile/water (1:1).

The detoxification test solution was prepared by mixing 900 μl 11.1 mM STS solution with 100 μl quenched linker-drug solution (quenched linker-drug 1 μM; STS 10 mM). LC-MS analysis was performed at the start of the experiment and after various time intervals.

No reaction occurred between quenched linker-drug and thiosulfate within the tested time frame of 24 hours. As thiosulfate does not react with quenched linker-drug, it is unlikely that vc-seco-DUBA and/or ADCs comprising this linker-drug will be affected in vivo.

Experiments in PBS buffer

Similar experiments were carried out with SYD986 and quenched linker-drug in Phosphate Buffered Saline (PBS) (Ix) buffer. SYD986 was tested at 0.5 μg/ml and 0.05 μg/ml (1.0 and 0.1 μM). Quenched linker-drug was tested at 1.5 μg/ml and 0.15 μg/ml (1.0 and 0.1 μM).

1x PBS has a final concentration of 137 mM NaCl, 10 mM phosphate, 2.7 mM KCl, and a pH of7.4.

The results in PBS were comparable to those in acetonitrile/water (1:1) described above.

Example 2—In vitro detoxification experiments with STS

Cell viability assay in SK-BR-3 cells

SK-BR-3 cells were exposed for 6 days to cyclopropyl-containing compound SYD986 or ADC SYD1875 in the presence or absence of 1 mM STS. SYD1875 is an ADC comprising an anti-5T4 antibody and linker-drug vc-seco-DUBA (ClinicalTrials.gov NCT04202705).

SK-BR-3 cells in complete growth medium were plated in 96-well plates at 6500 cells per well (90 μl/well) and incubated at 37° C., 5% CO2. After overnight incubation, 10 μl of SYD986 or SYD1875 with or without STS was added. Serial dilutions were made in culture medium. In the fixed 1 mM STS concentration experiments, a pre-mixture of a dilution range of SYD986 or SYD1875 concentrations was made and added to a fixed concentration of STS before addition to the cells.

Cell viability was assessed after 6 days using the PrestoBlue^{TM} cell viability assay and CyQUANT^{TM} direct cell proliferation assay from Invitrogen according to the manufacturer's instructions. Percentage survival was calculated by dividing the measured fluorescence for each SYD986 or SYD1875 concentration in the presence or absence of STS with the average mean of untreated cells multiplied by 100. Untreated cells were exposed to the appropriate vehicle only, i.e., growth medium with 1% DMSO+0.25% H20 in experiments with SYD986 (with or without STS), growth medium with 0.25% H20 in experiments with SYD1875 in the presence of STS, and growth medium only in experiments with SYD1875 without STS.

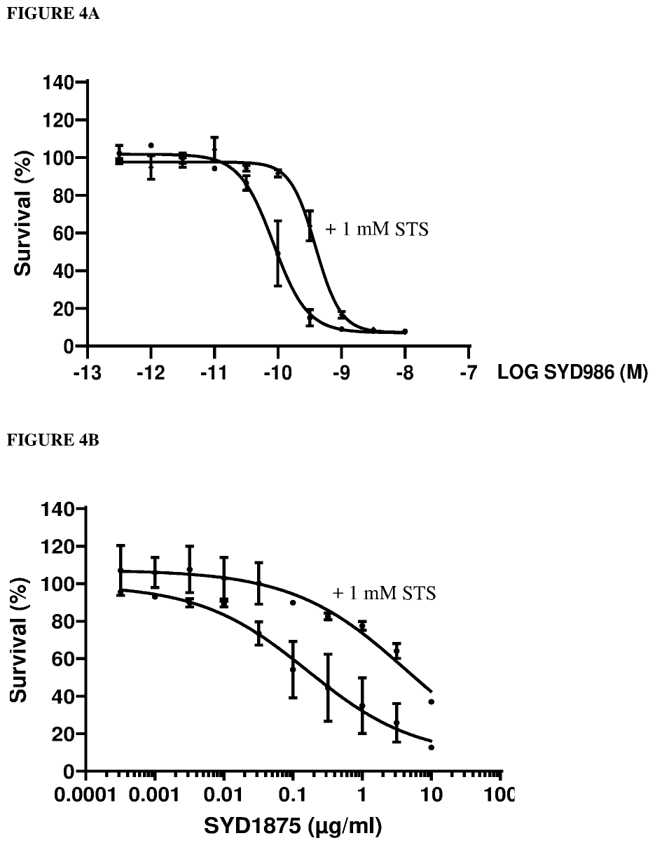

As can be seen in FIG. 4A, 1 mM STS negatively affects the potency of SYD986 in vitro in SK-BR-3 cells, thus showing that STS can detoxify the active duocarmycin drug. There is a 4.6-fold potency shift.

As can be seen in FIG. 4B, 1 mM STS negatively affects the potency of the anti-5T4 ADC SYD1875 in vitro in SK-BR-3 cells, thus showing that STS can detoxify the active duocarmycin drug after intracellular release. There is a 27-fold potency shift.

Example 3—In vivo safety experiments with STS administered via ocular route

A local tolerance and toxicity study in New Zealand White rabbits with formulations up to a concentration of 0.3 M STS did not show adverse findings.

A suitable protocol to confirm that STS is well tolerated in the eye in humans is outlined below:

Healthy subjects may be required to self-administer thiosulfate comprising eye drops in both eyes for 14 days. Each subject administers the eye drops once during Day 1, 3 times during Day 2 (starting in the morning every 2-3 hours) and 6 times daily (every 2-3 hours during waking hours) in the subsequent 12 days. Per administration subjects should apply one drop in each eye. Concentrations of 0.02, 0.05, 0.10 and 0.20 M STS may be used, formulated as detailed in Table 1-4.

TABLE 1
Composition comprising 0.02 M STS

| | Per vial | Concentration | | |
|---|---|---|---|---|
| Ingredients | (mg) | (mg/ml) | Quality | Function |
| Sodium thiosulfate | 2.48 | 4.96 | Ph. Eur./ | Active |
| pentahydrate | | | USP-NF | ingredient |
| Sodium hyaluronate | 0.50 | 1.0 | Ph. Eur. | Viscosity |
| 2000-2200 kDa | | | | agent |
| Sodium chloride | 3.6 | 7.2 | Ph. Eur./ | Isotonic |
| | | | USP-NF | agent |
| Sodium borate | 0.191 | 0.381 | Ph. Eur./ | Buffering |
| decahydrate | | | USP-NF | agent |
| Hydrochloric acid | q.s. to pH 7.4 | q.s. to pH 7.4 | Ph. Eur./ | PH |
| | | | USP-NF | adjustment |

| | |
|---|---|
| Total | 0.51 g |

q.s. = quantum satis

TABLE 2
Composition comprising 0.05 M STS

| | Per vial | Concentration | | |
|---|---|---|---|---|
| Ingredients | (mg) | (mg/ml) | Quality | Function |
| Sodium thiosulfate | 6.21 | 12.4 | Ph. Eur./ | Active |
| pentahydrate | | | USP-NF | ingredient |
| Sodium hyaluronate | 0.50 | 1.0 | Ph. Eur. | Viscosity |
| 2000-2200 kDa | | | | agent |
| Sodium chloride | 2.25 | 4.5 | Ph. Eur./ | Isotonic |
| | | | USP-NF | agent |
| Sodium borate | 0.191 | 0.381 | Ph. Eur./ | Buffering |
| decahydrate | | | USP-NF | agent |
| Hydrochloric acid | q.s. to pH 7.4 | q.s. to pH 7.4 | Ph. Eur./ | PH |
| | | | USP-NF | adjustment |

| | |
|---|---|
| Total | 0.51 g |

q.s. = quantum satis

TABLE 3
Composition comprising 0.10 M STS

| | Per vial | Concentration | | |
|---|---|---|---|---|
| Ingredients | (mg) | (mg/ml) | Quality | Function |
| Sodium thiosulfate | 12.4 | 24.8 | Ph. Eur./ | Active |
| pentahydrate | | | USP-NF | ingredient |
| Sodium hyaluronate | 0.50 | 1.0 | Ph. Eur. | Viscosity |
| 2000-2200 kDa | | | | agent |
| Sodium borate | 0.191 | 0.381 | Ph. Eur./ | Buffering |
| decahydrate | | | USP-NF | agent |
| Hydrochloric acid | q.s. to pH 7.4 | q.s. to pH 7.4 | Ph. Eur./ | PH |
| | | | USP-NF | adjustment |

| | |
|---|---|
| Total | 0.51 g |

q.s. = quantum satis

TABLE 4
Composition comprising 0.20 M STS

| | Per vial | Concentration | | |
|---|---|---|---|---|
| Ingredients | (mg) | (mg/ml) | Quality | Function |
| Sodium thiosulfate | 24.8 | 49.6 | Ph. Eur./ | Active |
| pentahydrate | | | USP-NF | ingredient |
| Sodium hyaluronate | 0.50 | 1.0 | Ph. Eur. | Viscosity |
| 2000-2200 kDa | | | | agent |
| Sodium borate | 0.191 | 0.381 | Ph. Eur./ | Buffering |
| decahydrate | | | USP-NF | agent |
| Hydrochloric acid | q.s. to pH 7.4 | q.s. to pH 7.4 | Ph. Eur./ | PH |
| | | | USP-NF | adjustment |

| | |
|---|---|
| Total | 0.52 g |

q.s. = quantum satis

Example 4—Protocol for Administration of STS Via Ocular Route to Diminish Potential Non-Target Ocular Toxicity of a Duocarmycin Derivative-Comprising Antibody-Drug Conjugate

The following protocol illustrates the way STS can be used in a clinical setting. By administering STS via ocular route (e.g. as eye drops) the potential non-target ocular toxicity of a duocarmycin derivative-comprising antibody-drug conjugate can be diminished.

STS containing eye drops, e.g. the eye drop formulations with 0.10 M or 0.20 M STS as exemplified in Tables 3 and 4 in Example 3, may be (self-)administered concurrently with treatment with SYD985, an ADC of vc-seco-DUBA and the antd-HER2 antibody trastuzumab.

When SYD985 is administered at a dose of 1.2 mg/kg body weight every three weeks, the STS eye drops can be used up to 6 times daily (approximately every 2 to 3 hours during waking hours) from the day of first infusion of SYD985 until 21 days after the last infusion or until the decision is made to discontinue SYD985 treatment, whichever is later.

1. A method for the treatment of a tumor in a human, which comprises administering to said human an antibody-drug conjugate in combination with thiosulfate;
wherein the ADC is a compound of formula (I) 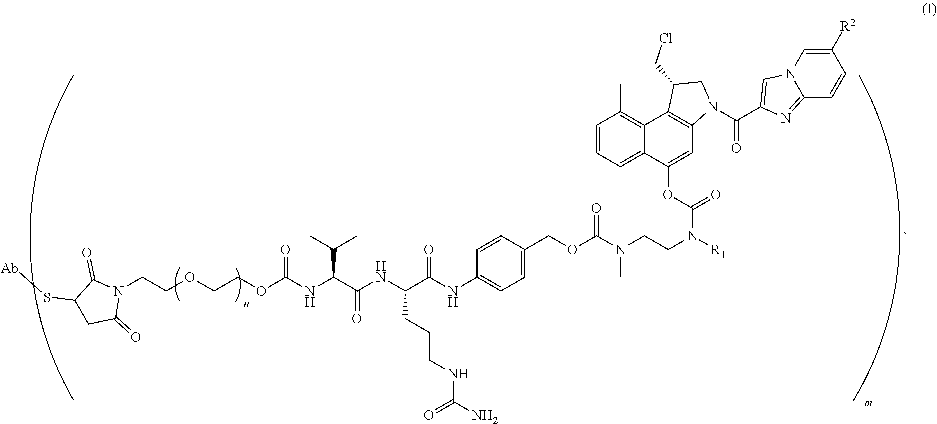
wherein
Ab is an antibody or an antigen-binding fragment of an antibody;
n is, 1,2 or 3;
m represents an average drug-to-antibody ratio (DAR) of from 1 to 6;
R¹ is selected from the group consisting of 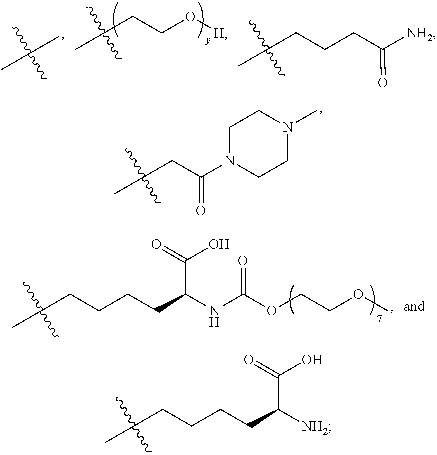
y is an integer of from 1-16; and
R² is selected from the group consisting of

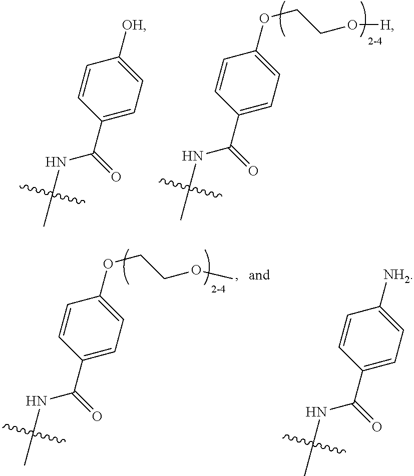

2. The method An ADC feruse-according to claim 1, wherein
n is0or 1;
m represents an average DAR of from 1 to 4;
R¹ is 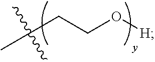
y is an integer of from 1-4; and
R² is selected from the group consisting of

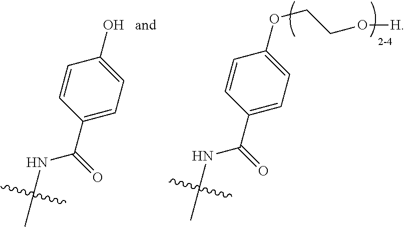

3. The method according to claim 1, wherein the ADC is a compound of formula (II)

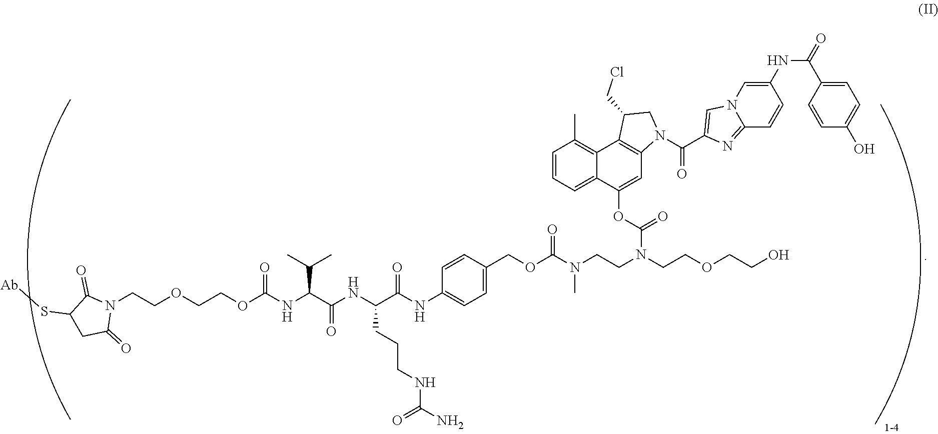

4. The method according to claim 1, wherein the thiosulfate is sodium thiosulfate (STS).

5. (canceled)

6. The method according to claim 3, wherein the thiosulfate is STS.

7. (canceled)

8. (canceled)

9. The method according to claim 1 wherein the ADC and the thiosulfate are administered simultaneously, separately or sequentially.

10. The method according to claim 1, wherein the thiosulfate is administered by inhalation or via intravenous, oral, dermal, subcutaneous or ocular route.

11. (canceled)

12. (canceled)

13. The method according to claim 10, wherein the thiosulfate is administered via ocular route.

14. A method for preventing or reducing toxicity associated with the administration of an antibody-drug conjugate (ADC) of formula (I) 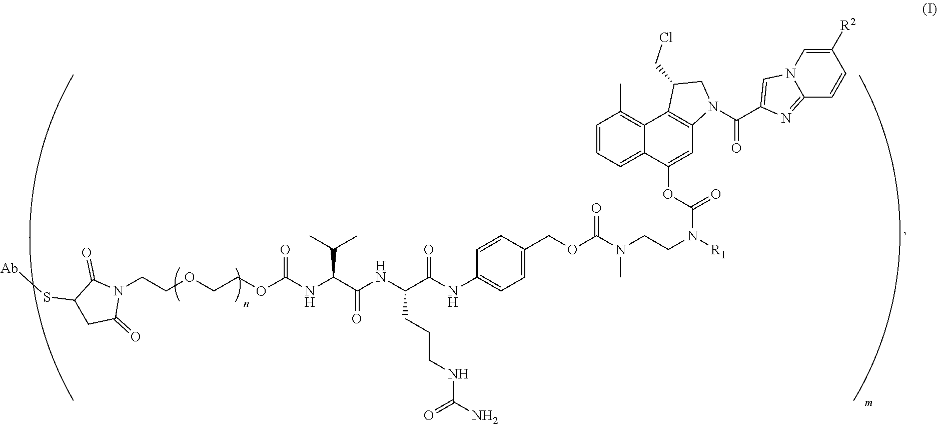
wherein
Ab is an antibody or an antigen-binding fragment of an antibody; ^{PGP}-5⁸,c¹
n is0, 1,2 or3:
m represents an average drug-to-antibody ratio (DAR) of from 1 to 6:
R¹ is selected from the group consisting of 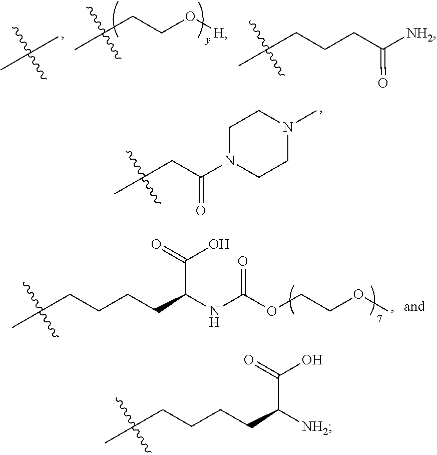
y is an integer of from 1-16: and
R² is selected from the group consisting of 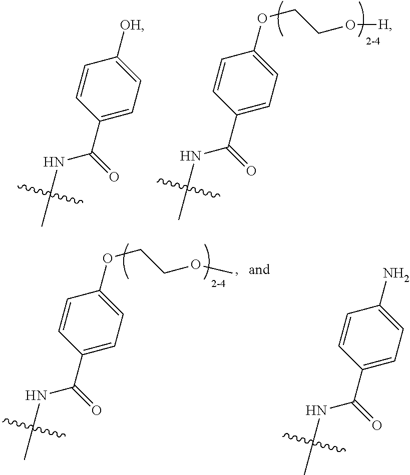
comprising administering an effective amount of the ADC of formula (I) in combination with an effective amount of thiosulfate, preferably-wherein the thiosulfate is administered from about three weeks before to about 1 hour after the first administration of the ADC and the administration of thiosulfate is repeated at regular intervals until up to three months after the last administration of the ADC.

15. The method according to claim 14, wherein the thiosulfate is administered by inhalation or via intravenous, oral, dermal, subcutaneous or ocular route.

16. The method according to claim 15, wherein said thiosulfate is STS.

17. In a method of treating a tumor in a human that comprises administering to the human a duocarmycin derivative-comprising ADC having the formula Ab-(L-D)ₘ,
wherein Ab is an antibody or an antigen-binding fragment of an antibody, L-D is a duocarmycin derivative linker-drug and m represents an average DAR of from 1 to 12, and
wherein the duocarmycin derivative drug consists of a DNA-binding (DB) moiety and a DNA-alkylating (DA) moiety as represented by formula (IV) 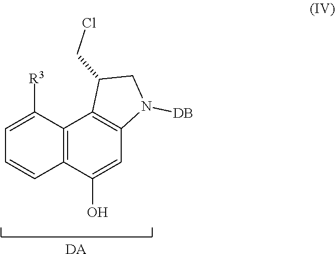
wherein DB is selected from 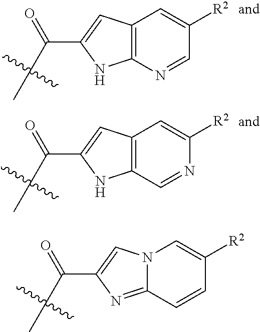
wherein R² and R³ are independently selected from H, OH, SH, NH₂, N₃, NO₂, NO, CF₃, CN, C(O)NH₂, C(O)H, C(O)OH, halogen, Ra, SRI, S(O)Ra, S(O)₂Ra, S(O)ORa, S(O)₂ORa, OS(O)Ra, OS(O)₂Ra, OS(O)ORa, OS(O)₂ORa, ORe, NHRa, N(Ra)Rb, ⁺N(RaXRb)Rc, P(OXORaXORb), OP(OXORaXORb), SiRaRbRC, C(O)Ra, C(O)ORa, C(O)N(Ra)Rb, OC(O)Ra, OC(O)ORa, OC(O)N(Ra)Rb, N(Ra)C(O)Rb, N(Ra)C(O)OR^{b}, N(Ra)C(O)N(R^{b})Rc, and a water-soluble group; and
wherein R^{a}, R^{b}, and R^{c} are independently selected from H and optionally substituted (CH₂CH₂O)ₐₐCH₂CH₂X¹R^{a1} , C₁₋₁₅ alkyl, C₁₋₁₅ heteroalkyl, C₃₋₁₅ cycloalkyl, C₁₋₁₅ heterocycloalkyl, C₅₋₁₅ aryl, or C₁₋₁₅ heteroaryl, wherein aa is selected from 1 to 1000, X¹ is selected from O, S, and NR^{b1} , and R^{b1} and R^{a1} are independently selected from H and C₁₋₃ alkyl, one or more of the optional substituents in R^{a}, R^{b}, and/or RC optionally being a water-soluble group, two or more of R^{a}, R^{b}, and RC optionally being joined by one or more bonds to form one or more optionally substituted carbocycles and/or heterocycles;
wherein the improvement comprises further administering to said human a thiosulfate in an amount effective to prevent or treat unwanted non-target tissue toxicity.

18. The method according to claim 17, wherein said thiosulfate is STS.

19. The method according to claim 18, wherein said thiosulfate is administered by inhalation, or via intravenous, oral, dermal, subcutaneous or ocular route.

20. The method according to claim 19, wherein said thiosulfate is administered via intravenous and subsequently subcutaneous routes.

21. The method according to claim 19, wherein said thiosulfate is administered via an ocular route.

22. The method according to claim 18, wherein said duocarmycin derivative-comprising ADC is a compound of formula (I) 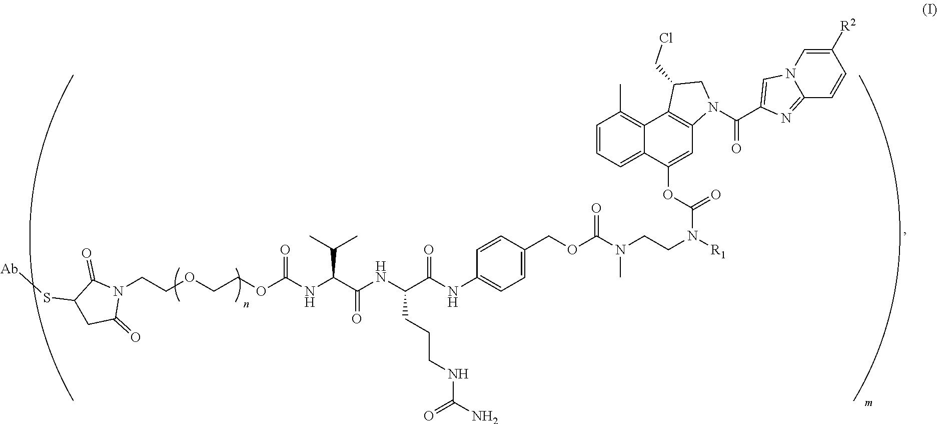
wherein
Ab is an antibody or an antigen-binding fragment of an antibody;
n is 0, 1,2or3;
m represents an average drug-to-antibody ratio (DAR) of from 1 to 6;
R¹ is selected from the group consisting of 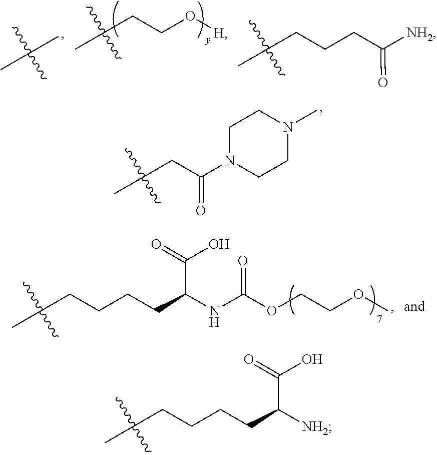
y is an integer of from 1-16; and
R² is selected from the group consisting of

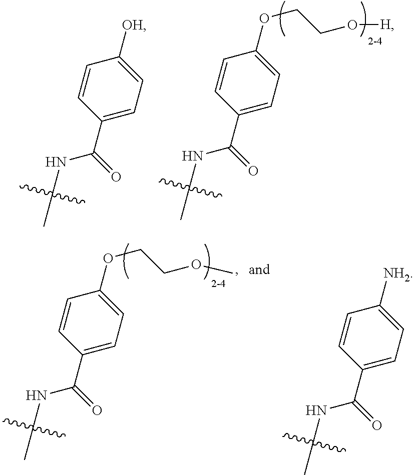

23. The method according to claim 22, wherein said ADC is a compound of formula (II)

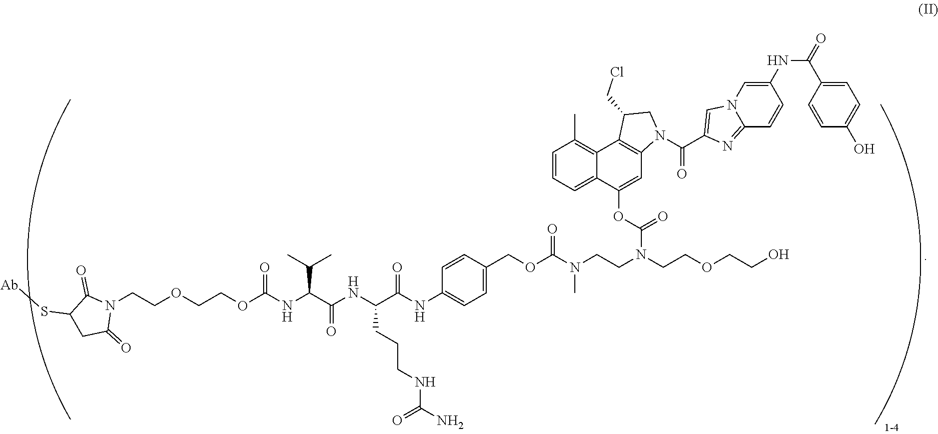

24. The method according to claim 23, wherein Ab is trastuzumab. PGP-,

25. The method according to claim 18, wherein Ab exhibits binding to a tumor associated antigen (TAA).
